SEQUENCE LISTING (78 sequences)

SEQ ID NO: 1 - DNA, 20 bp, Artificial Sequence
  Description of Artificial Sequence Synthetic primer
atgaccatgattacgccaag

SEQ ID NO: 2 - DNA, 19 bp, Artificial Sequence
  Description of Artificial Sequence Synthetic primer
gctagccaagggtcctcct

SEQ ID NO: 3 - DNA, 43 bp, Artificial Sequence
  Description of Artificial Sequence Synthetic primer
aggaggacccttggctagcatgacagcttcatcttctgggtgt

SEQ ID NO: 4 - DNA, 53 bp, Artificial Sequence
  Description of Artificial Sequence Synthetic primer
taatcagtatgtttattgaaatgaaagatagaaaatcatgagttgtattcgtg

SEQ ID NO: 5 - DNA, 37 bp, Artificial Sequence
  Description of Artificial Sequence Synthetic primer
aggaggacccttggctagcatgcttggatcccgcttc

SEQ ID NO: 6 - DNA, 22 bp, Artificial Sequence
  Description of Artificial Sequence Synthetic primer
aacaggcgtcggttctttattt

SEQ ID NO: 7 - DNA, 40 bp, Artificial Sequence
  Description of Artificial Sequence Synthetic primer
aggaggacccttggctagcatgatgacgtcactgctcact

SEQ ID NO: 8 - DNA, 22 bp, Artificial Sequence
  Description of Artificial Sequence Synthetic primer
ttattttctgccaaaacgaatg

SEQ ID NO: 9 - DNA, 48 bp, Artificial Sequence
  Description of Artificial Sequence Synthetic primer
aggaggacccttggctagcatgttgtcgtcgcgcacatcgtccatcat

SEQ ID NO: 10 - DNA, 23 bp, Artificial Sequence
  Description of Artificial Sequence Synthetic primer
tcagatgcttctttttccaaatc

SEQ ID NO: 11 - DNA, 58 bp, Artificial Sequence
  Description of Artificial Sequence Synthetic primer
gtgctgcaccagcctcagcggtttggaaaaagaagcatctgataatataccatctacc

SEQ ID NO: 12 - DNA, 60 bp, Artificial Sequence
  Description of Artificial Sequence Synthetic primer
gatgcttctttttccaaaccgctgaggctggtgcagcacacgtttgtgtggaatctctcc

SEQ ID NO: 13 - DNA, 57 bp, Artificial Sequence
  Description of Artificial Sequence Synthetic primer
ccagctcacctgcatgcaaacctccctcttcgctttggaaaaagaagcatctgataa

SEQ ID NO: 14 - DNA, 55 bp, Artificial Sequence
  Description of Artificial Sequence Synthetic primer
agcgaagagggaggtttgcatgcaggtgagctggacgtttgtgtggaatctctcc

SEQ ID NO: 15 - DNA, 19 bp, Artificial Sequence
  Description of Artificial Sequence Synthetic primer
ctagcaaaaatgcataagg

SEQ ID NO: 16 - DNA, 22 bp, Artificial Sequence
  Description of Artificial Sequence Synthetic primer
gtgtcagaggttttcaccgtca

SEQ ID NO: 17 - DNA, 41 bp, Artificial Sequence
  Description of Artificial Sequence Synthetic primer
ctgtaatttttaactttcagatggtctcaaagggtgaagaa

SEQ ID NO: 18 - DNA, 28 bp, Artificial Sequence
  Description of Artificial Sequence Synthetic primer
ctacttatacaattcatccatgccacct

SEQ ID NO: 19 - DNA, 19 bp, Artificial Sequence
  Description of Artificial Sequence Synthetic primer
ctagcaaaaatgcataagg

SEQ ID NO: 20 - DNA, 25 bp, Artificial Sequence
  Description of Artificial Sequence Synthetic primer
tttttctaccggtaccttacgcttc

SEQ ID NO: 21 - DNA, 23 bp, Artificial Sequence
  Description of Artificial Sequence Synthetic primer
catcgtcgtaaaaacaaattcaa

SEQ ID NO: 22 - DNA, 39 bp, Artificial Sequence
  Description of Artificial Sequence Synthetic primer
ccttatgcatttttgctagtttgacacaaaatgccgact

SEQ ID NO: 23 - DNA, 24 bp, Artificial Sequence
  Description of Artificial Sequence Synthetic primer
catccaatatggtgagtttctctg

SEQ ID NO: 24 - DNA, 45 bp, Artificial Sequence
  Description of Artificial Sequence Synthetic primer
ccttatgcatttttgctagcgtctgaaatttcgaaaagtaataat

SEQ ID NO: 25 - DNA, 25 bp, Artificial Sequence
  Description of Artificial Sequence Synthetic primer
agttttgaagcttacttgacatgaa

SEQ ID NO: 26 - DNA, 39 bp, Artificial Sequence
  Description of Artificial Sequence Synthetic primer
ccttatgcatttttgctagagggaagggaaaaacttgaa

SEQ ID NO: 27 - DNA, 24 bp, Artificial Sequence
  Description of Artificial Sequence Synthetic primer
tgttttcaaagtgttggaatcaat

SEQ ID NO: 28 - DNA, 39 bp, Artificial Sequence
  Description of Artificial Sequence Synthetic primer
aaaaatcgatcctgcaggcgaaccgaaccgaatgaaaca

SEQ ID NO: 29 - protein, 11 aa, Caenorhabditis elegans
SPMQRSSMVRF

SEQ ID NO: 30 - protein, 11 aa, Caenorhabditis elegans
TPMQRSSMVRF

SEQ ID NO: 31 - protein, 11 aa, Caenorhabditis elegans
SPMERSAMVRF

SEQ ID NO: 32 - protein, 11 aa, Caenorhabditis elegans
SPMDRSKMVRF

SEQ ID NO: 33 - protein, 7 aa, Caenorhabditis elegans
KPSFVRF

SEQ ID NO: 34 - protein, 9 aa, Caenorhabditis elegans
AMDSPLIRF

SEQ ID NO: 35 - protein, 10 aa, Caenorhabditis elegans
AADGAPLIRF

SEQ ID NO: 36 - protein, 10 aa, Caenorhabditis elegans
APEASPLIRF

SEQ ID NO: 37 - protein, 10 aa, Caenorhabditis elegans
ASPSAPLIRF

SEQ ID NO: 38 - protein, 10 aa, Caenorhabditis elegans
SPSAVPLIRF

SEQ ID NO: 39 - protein, 9 aa, Caenorhabditis elegans
ASSAPLIRF

SEQ ID NO: 40 - protein, 9 aa, Caenorhabditis elegans
SAAAPLIRF

SEQ ID NO: 41 - protein, 9 aa, Caenorhabditis elegans
SPSAKWMRF

SEQ ID NO: 42 - protein, 11 aa, Caenorhabditis elegans
VPSAGDMMVRF

SEQ ID NO: 43 - protein, 160 aa, Caenorhabditis elegans
MMTSLLTISMFVVAIQAFDSSEIRMLDEQYDTKNPFFQFLENSKRSDRPTRAMDSPLIRFGKRAADGAPLIRFGRAPEASPFIRFGKRAADGAPLIRFGRAPEASPFIRFGKRASPSAPLIRFGRSPSAVPLIRFGRSAAAPLIRFGRASSAPLIRFGRK

SEQ ID NO: 44 - protein, 160 aa, Caenorhabditis brenneri
MMTSLLVIPMMFVVAIQAFDSSEIRMLDEQYETNHPYFPFLEQKRSDRPTRAMDSPLIRFGKRAADGAPLIRFGRAPEASPFIRFGKRAADGAPLIRFGRAPEASPFIRFGKRAAPSAPLIRFGRSPSAAPLIRFGRSAAAPLIRFGRASSAPLIRFGRK

SEQ ID NO: 45 - protein, 161 aa, Caenorhabditis remanei
MMTPFLVISLMFVVSIQAFDSSEIRMLDEQYEPKNPYFPFLENEKRSDRPTRAMDSPLIRFGKRAADGAPLIRFGRAPEASPFIRFGKRAADGAPLIRFGRAPEASPFIRFGKRASPSAPLIRFGRSPSAVPLIRFGRSAAAPLIRFGRASSAPLIRFGRK

SEQ ID NO: 46 - protein, 160 aa, Caenorhabditis briggsae
MMTSLVFAMMFAVAAVQAFDSSEIRMLDEQYDKSPYFPFLENEKRSDRPTRAMDSPLIRFGKRAADGAPLIRFGRAPEASPFIRFGKRAADGAPLIRFGRAPEASPFIRFGKRASPSAPLIRFGRSPSSAPLIRFGRSAGAPLIRFGRASSAPLIRFGRK

SEQ ID NO: 47 - protein, 152 aa, Caenorhabditis japonica
MMTSLAVLLVVFVATSEAFDSSEIRMLDEQYDTKNPFLAYLENQKRSDRPTRALDSLDSPLIRFGKRAADGAPLIRFGRAPEASPFIRFGKRASAVPLIRFGRAPEASPFIRFGKRASPSAPLIRFGRSPAAVPLIRFGRSAAAPLIRFGRK

SEQ ID NO: 48 - protein, 137 aa, Ascaris suum
MAINSISCLITMLAITVAVSTLESSEIRMLESEFLDGKRGNSYRSFFNPNEYTVVERRDSKLMDPLIRFGKRTNIMGENRLNRNLRAEGLSSPLIRFGKRTPPEEDLLGRFTRDPQQRIVTDETVLRFGRSSAPAAA

SEQ ID NO: 49 - protein, 69 aa, Caenorhabditis elegans
MLSSRTSSIILILAILVAIMAVAQCRNIQYDVEEMTPEAAFRYAQWGEIPHKRVPSAGDMMVRFGKRSI

SEQ ID NO: 50 - protein, 68 aa, Caenorhabditis brenneri
MISSRSSIILILAILVAIMAVAQCRNIQYDIDEIAPESAFRYAQWGEIPHKRVPSAGDMMVRFGKRAV

SEQ ID NO: 51 - protein, 68 aa, Caenorhabditis remanei
MLSSRASIVLIFAILVAIMAVAQCRNIQYDIDEITPEAAFRYAQWGEIPHKRVPSAGDMMVRFGKRSV

SEQ ID NO: 52 - protein, 66 aa, Caenorhabditis briggsae
MSRTSIILVLAIFVAIAAIAQCRNIQYDVDEISPEAAFRYAQWGEIPHKRVPSAGDMMVRFGKRSV

SEQ ID NO: 53 - protein, 62 aa, Pristionchus pacificus
MKSILIILLSFILLLSLSSARNIFPYGPVGQMGEINAEEREWMIPSMAKRVPSAGDMMVRFG

SEQ ID NO: 54 - protein, 81 aa, Ascaris suum
MFSLKAIVMIALVVICTFCISESRRFHDDDFSRQFLFRGIDEPLKNYMRLREARILSKRVPSAADMMIRFGKRSFIEQDME

SEQ ID NO: 55 - protein, 172 aa, Caenorhabditis elegans
MSQKLLPISPLQLLFLQCLLIGFTAAYPYLIFPASPSSGDSRRLVKRAFDRFDNSGVFSFGAKRFDRYDDETAYGYGFDNHIFKRSADPYRFMSVPTKKAFDRMDNSDFFGAKRKRSFDRMGGTEFGLMKRSAPESREQLINNLAESIITLRRAREAESSPESQRTIITYDD

SEQ ID NO: 56 - protein, 172 aa, Caenorhabditis briggsae
MSQKLLSISPLRLLFLQCLLIGFAVSYPYLIFPASPSSGDSRRLVKRAFDRFDNSGVFSFGSKRFDRYEEENPYGYNFGAHIFKRSADPYRFMTVPTKKAFDRMDNSDFFGAKRKRSFDRMGGTEFGLMKRSAPEDREQLIDSLTDSILALRRARESESSGTTQPVAISYED

SEQ ID NO: 57 - protein, 170 aa, Caenorhabditis brenneri
MSQKLLPISPLQLLLLQCLLIGYVMAYPYLIVPSSPSGDSRRLVKRAFDRFDNSGVFSFGAKRFDRYDDENPYGYVYDSHIFKRSADPYRFMTVPTKKAFDRMDTNDFFGAKRKRSFDRMGGTEFGLMKRSGGTENREQLIDNLTESIIALRRARETEGGSQPVVVSYED

SEQ ID NO: 58 - protein, 170 aa, Caenorhabditis remanei
MSQKLLPISPLRLLLLQCILIGFTVAYPYLIFPASSASSGDSRRLVKRAFDRFDNSGVFSFGAKRFDRYDEENPYGYNYNTHIYKRSADPMRFVSMPAKKAFDRMDASDFFGAKRKRSFDRMGGTEFGLMKRSVPENREQLIDSLTDSIMALRKARESEGSQPVLVNFED

SEQ ID NO: 59 - protein, 172 aa, Caenorhabditis japonica
MSEKHPILPVSPIRLLLLQFFVLGVAAAYPYLLLPTSSDVGSRRLVKRAFDRFDNSGVFAFGAKRFDDFQDDSYGYGYPDGSQIFKRSVDPSRFVTVPSKKAFDRMDGGVDFFGAKRKRAFDRLGGTEFGLVKRSTGDREKLIDNLTESIIALRKSRAAEGGSQPVVVTYDD

SEQ ID NO: 60 - protein, 9 aa, Caenorhabditis elegans
AMDSPLIRF

SEQ ID NO: 61 - protein, 10 aa, Caenorhabditis elegans
AADGAPLIRF

SEQ ID NO: 62 - protein, 10 aa, Caenorhabditis elegans
APEASPFIRF

SEQ ID NO: 63 - protein, 10 aa, Caenorhabditis elegans
ASPSAPLIRF

SEQ ID NO: 64 - protein, 10 aa, Caenorhabditis elegans
SPSAVPLIRF

SEQ ID NO: 65 - protein, 9 aa, Caenorhabditis elegans
SAAAPLIRF

SEQ ID NO: 66 - protein, 9 aa, Caenorhabditis elegans
ASSAPLIRF

SEQ ID NO: 67 - protein, 10 aa, Danio rerio
HLHANLPLRF

SEQ ID NO: 68 - protein, 8 aa, Danio rerio
VPNLPQRF

SEQ ID NO: 69 - protein, 11 aa, Caenorhabditis elegans
VPSAGDMMVRF

SEQ ID NO: 70 - protein, 180 aa, Homo sapiens
MEIISSKLFILLTLATSSLLTSNIFCADELVISNLHSKENYDKYSEPRGYPKGERSLNFEELKDWGPKNVIKMSTPAVNKMPHSFANLPLRFGRNVQEERSAGATANLPLRSGRNMEVSLVRRVPNLPQRFGRTTTAKSVCRMLSDLCQGSMHSPCANDLFYSMTCQHQEIQNPDQKQSR

SEQ ID NO: 71 - protein, 188 aa, Mus musculus
MEIISLKRFILLTVATSSFLTSNTFCTDEFMMPHFHSKEGDGKYSQLRGIPKGEKERSVSFQELKDWGAKNVIKMSPAPANKVPHSAANLPLRFGRTIDEKRSPAARVNMEAGTRSHFPSLPQRFGRTTARSPKTPADLPQKPLHSLGSSELLYVMICQHQEIQSPGGKRTRRGAFVETDDAERKPEK

SEQ ID NO: 72 - protein, 173 aa, Gallus gallus
MEVISTQKFILLTLATVAFLTPHGMCLDELMKSSLESREEDDDKYYEIKDSILEEKQRSLNFEEMKDWGSKNFLKVNTPTVNKVPNSVANLPLRFGRSNPEERSIRPSAYLPLRFGRAFGESLSRRAPNLSYRLGRSPLARSSIQSLLNLPQRFGKSVPINLSQGVQESEPGM

SEQ ID NO: 73 - protein, 197 aa, Xenopus tropicalis
RHNNMEMISASKVILFYLSTYAIFFISSIYSAENTGNSMENQENDDGLLKPTDDIQNPRTINSEELQYWKSNIQNELDSLLAYKYNNLPLQSERLVLEERDNKPAANLPLRFGRGSEDGITRSIPNLPQRFGRYVPAKANIPSLANLPQRFGRSSQGGRYVQSLATLPQRFGRETPLQRLQYGMKPYLQEVRSPDDD

SEQ ID NO: 74 - protein, 198 aa, Danio rerio
MSYFALLSLALGILSSFMLSEVTALRLPLSGERDLNGFTWGQFSENAQEIPRSLEIQDFTLNVAPTSGGASSPTILRLHPIIPKPAHLHANLPLRFGRDAQPGTGDRAPKSTINLPQRFGRSCTMCARSGTGPSATLPQRFGRRNIFALDPLRALALYTRTPESPSFPKERTQVHDYMFETVEDSEETVKNTDYTALD

SEQ ID NO: 75 - protein, 173 aa, Takifugu rubripes
MLVTAFLGMLLMIAGIGGAAETDLQVNGKLNDRTLSSREGRHNVRKQLRHQIKSNILRSLDMERINIQVSPTSGKVSLPTIVRLYPPTLQPHHQHVNMPMRFGRDGVQGGDHVPNLNPKMPQRFGRSWKVIRLCEDCSKVQGVLKHQVRYGRNGQSLIRTLVNAQLLKTGLHW

SEQ ID NO: 76 - protein, 12 aa, Homo sapiens
  C-term amidated
MPHSFANLPLRF

SEQ ID NO: 77 - protein, 12 aa, Homo sapiens
  C-term amidated
SAGATANLPLRS

SEQ ID NO: 78 - protein, 8 aa, Homo sapiens
  C-term amidated
VPNLPQRF

(19) United States
(12) Patent Application Publication
(10) Pub. No.: US 2015/0246140 A1
(43) Pub. Date: Sep. 3, 2015

(54) COMPOSITIONS AND METHODS FOR MODULATING SLEEP AND WAKEFULNESS

(71) Applicants: Suk Hen Chow, San Marino, CA (US); Daniel A. Lee, Pasadena, CA (US); David A. Prober, Pasadena, CA (US); Paul W. Sternberg, Pasadena, CA (US)

(72) Inventors: Suk Hen Chow, San Marino, CA (US); Daniel A. Lee, Pasadena, CA (US); David A. Prober, Pasadena, CA (US); Paul W. Sternberg, Pasadena, CA (US)

(73) Assignee: California Institute of Technology, Pasadena, CA (US)

(21) Appl. No.: 14/618,523

(22) Filed: Feb. 10, 2015

Related U.S. Application Data
(60) Provisional application No. 61/938,046, filed on Feb. 10, 2014.

(51) Int. Cl. A61K 49/00

(52) U.S. Cl. CPC A61K 49/0008

(57) ABSTRACT

In some aspects, the invention relates to methods of treating conditions associated with sleep disorders, comprising administering an agent that modulates the activity of a neuropeptide receptor or a neuropeptide receptor ligand. In some aspects, the invention relates to methods of screening agents that modulate the activity of a neuropeptide receptor in an organism, optionally using cells or organisms comprising a genetic modification that affects a neuropeptide signaling pathway. In some aspects, the invention relates to methods of characterizing a condition associated with a sleep disorder comprising sequencing a nucleic acid encoding either a neuropeptide receptor or a neuropeptide receptor ligand and comparing the sequence with a reference sequence.

### PRIORITY CLAIM

This application claims priority to U.S. Provisional Patent Application No. 61/938,046, filed on Feb. 10, 2014, hereby incorporated by reference in its entirety.

### STATEMENT REGARDING FEDERALLY SPONSORED RESEARCH OR DEVELOPMENT

This invention was made with government support under NS070911, GM084389, and DA031367, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 24, 2015, is named CTH-01501_SL.txt and is 40,857 bytes in size.

### BACKGROUND

More than 30 million Americans suffer from chronic sleep disorders such as narcolepsy and insomnia. Safe, effective, non-addictive, and long-lasting drugs, however, are substantially unavailable. For example, a recent, large longitudinal study found that the use of sleep aids significantly increases patient mortality (Kripke, D. F. et al., British Medical Journal 2:e000850 (2012)). Barbiturates, for example, have addictive tendencies and can be lethal at high dosages. Benzodiazepines, such as temazepam, are less effective and tend to lose their sedative effect with continued use. Nonbenzodiazepines, such as the GABA_{A} receptor agonist zolpidem, are similarly less effective and also lose their sedative effect with continued use. In comparison, gammahydroxybutyrate (GHB) is a powerful muscle relaxant that is effective for treating narcolepsy, but GHB has a short duration of action of about two to three hours and a propensity for abuse. Despite the pressing need for new agents to treat sleep disorders, relatively little is known of the genetic circuits regulating sleep and wakefulness.

### SUMMARY

In some aspects, the invention relates to methods of treating conditions associated with sleep disorders, comprising administering an agent that modulates the activity of a neuropeptide receptor or a neuropeptide receptor ligand. In some aspects, the invention relates to methods of screening for agents that modulate the activity of a neuropeptide receptor in an organism, optionally using cells or organisms comprising a genetic modification that affects a neuropeptide signaling pathway. In some aspects, the invention relates to methods of characterizing a condition associated with a sleep disorder comprising sequencing a nucleic acid encoding either a neuropeptide receptor or a neuropeptide receptor ligand and comparing the sequence with a reference sequence.

### DESCRIPTION OF THE FIGURES

FIG. 1 includes six panels, identified as panels (A), (B), (C), (D), (E), and (F). Panel (A) depicts expression data of 8,113 genes collected from two pools of genes in ALA compared with mixed-stage larvae (Table 1). Wild type ALA versus larval expression ratio showed that eight neuropeptides have >17× expression in ALA (Table 1). flp-24 has the highest ALA/whole larvae ratio expression, followed by flp-7, flp-13 and nlp-8. Panel (B) depicts a schematic diagram of sleep assays that utilize locomotion, feeding, and sensory arousal behaviors as read outs. Panel (C) depicts the time course of feeding quiescence in wild type and mutants of ALA-synthesized neuropeptides after exposure to heat. Animals defective of ALA-synthesized neuropeptides are resistant to EFG-induced feeding quiescence. Panel (D) depicts feeding and locomotion quiescence, which are consistent with activation of EGF (hs:LIN-3C). Basal activity of heat-shock treatment is normalized by animals with no activated neuropeptide (No hs) and with an activated non-peptide (hs:HLH-13) and an activated non-functional peptide (hs:FLP-24PS). Panel (E) depicts a comparison of feeding between animals with and without activated neuropeptide by quantifying pharyngeal pumping per min (ppm) (C). Panel (F) shows that responses to 30% 1-octanol are increased in animals with heat-activated EGF and neuropeptides. Error bars in all figures indicate SEM. ***p<0.0001, **p<0.001, *p<0.05. N=100.

FIG. 2 depicts EGF induction of sleep through FLP-13 and FLP-24 neuropeptides. hs:FLP-13 or hs:FLP-24 is sufficient to restore sleep-inducing effect in ceh-14 mutants. Error bars indicate SEM. ***p<0.0001. N=100.

FIG. 3 includes four panels, identified as panels (A), (B), (C), and (D). Panel (A) depicts time courses of feeding quiescence in wild type compared with mutants of G-protein coupled receptors (GPCRs) after exposure to heat. Animals lacking npr-3, npr-7 and npr-22 exhibit resistance to sleep induction, demonstrated by reduced feeding quiescence. Panel (B) shows that feeding and locomotion quiescence is completely blocked in animals lacking npr-7 when activating FLP-13 and zebrafish NPSF peptides. The effect is partial in npr-22 mutants. Panel (C) shows that mutation of npr-7 restored acute response to 30% 1-octanoal in animals with activated FLP-13 and zebrafish NPSF peptides. Mutation of npr-22 partially restores the response to 30% 1-octanol in animals with activated FLP-13 and FLP-24 peptides. Effects were compared with wild type animals with no activated neuropeptide. Error bars in all figures indicate SEM. ***p<0.0001, **p<0.001, *p<0.05. N=100.

FIG. 4 includes six panels, identified as panels (A), (B), (C), (D), (E), and (F). Panel (A) depicts zebrafish sleep/wake behavior in wild-type and hs:RFRP siblings. HS (vertical bar) denotes where fish were moved from the videotracker to a 37° C. incubator and heatshocked. Panel (B) shows that conditional heat-shock-induced RFRP overexpression (light grey bars) reduces locomotor activity and commensurately increases sleep/rest compared to wild type sibling controls (dark grey bars). Panels (C), (D), (E), and (F) depict average sleep (min/hour), sleep bout length, sleep latency, and wake bout length quantified for each time period pre- and post-HS. Data is represented as mean±SEM for 118 WT and 130 HS:RFRP transgenic fish averaged over 3 independent experiments. ***p<0.0001.

FIG. 5 includes four panels, identified as panels (A), (B), (C), and (D). Panel (A) shows mid-L4 larvae that were adhered to a freshly made agar plate with dental glue along the ventral bodyline. Panel (B) shows the identification of the ALA neuron as dorsal to the pharynx and labeled with a GFP reporter diving ceh-14 expression in the ALA neuron; this was the only dorsal head neuron expressing ceh-14::GFP. Panel (C) depicts a fine glass cutting needle (arrowhead) used to cut open the dorsal worm body close to the vulva to release body pressure and a small puncture in the dorsal head just big enough to release the ALA neuron. Panel (D) shows a glass patch needle (arrow), which was used to collect the releasing ALA neuron.

FIG. 6 shows that C. elegans flp-13 is a conserved nematode neuropeptide-coding precursor gene. flp-13 encodes a neuropeptide propeptide gene that generates 9 peptides (P1-P9), in which P2 and P4, P3 and P5, are repeated copies and have inhibitory effects (triangles) on A. suum muscle strips, while P7 has excitatory effect (square) on A. suum muscle strips. P2, P4, P6, P7, P8 and P9 can activate calcium response via NPR-22 using the fluorescence assay (diamonds). Neuropeptide annotation and sequence alignment were conducted by www.uniprot.org. Amino acid sequences for FLP-13 in nematodes are shown: Caenorhabditis elegans (O44185) (SEQ ID NO: 43), Caenorhabditis brenneri (G0P6W9) (SEQ ID NO: 44), Caenorhabditis remanei (E3M7H9) (SEQ ID NO: 45), Caenorhabditis briggsae (A8X1A3) (SEQ ID NO: 46), Caenorhabditis japonica (H2W239) (SEQ ID NO: 47), and Ascaris suum (D9I8P2) (SEQ ID NO: 48).

FIG. 7 shows that C. elegans flp-24 is a conserved nematode neuropeptide-coding precursor gene. flp-24 encodes a neuropeptide propeptide gene that generates 1 peptide. Neuropeptide annotation and sequence alignment were conducted by www.uniprot.org. Amino acid sequences for FLP-24 in nematodes are shown: Caenorhabditis elegans (044185) (SEQ ID NO: 49), Caenorhabditis brenneri (GOP6W9) (SEQ ID NO: 50), Caenorhabditis remanei (E3M7H9) (SEQ ID NO: 51), Caenorhabditis briggsae (A8X1A3) (SEQ ID NO: 52), Pristionchus pacificus (H3ENH6) (SEQ ID NO: 53), and Ascaris suum (Q5ENY8) (SEQ ID NO: 54).

FIG. 8 shows that C. elegans flp-8 is a conserved nematode neuropeptide-coding precursor gene. nlp-8 encodes a neuropeptide propeptide gene that generates 4 peptides with FDR N-terminal. Neuropeptide annotation and sequence alignment were conducted by www.uniprot.org. Amino acid sequences for NLP-8 in nematodes are shown: Caenorhabditis elegans (Q93409) (SEQ ID NO: 55), Caenorhabditis briggsae (A8X671) (SEQ ID NO: 56), Caenorhabditis brenneri (GOP745) (SEQ ID NO: 57), Caenorhabditis remanei (E3N7Q4) (SEQ ID NO: 58), and Caenorhabditis japonica (H2W434) (SEQ ID NO: 59).

FIG. 9 includes seven panels, identified as panels (A), (B), (C), (D), (E), (F), and (G). Panel (A) depicts the architecture of the GFP expression construct. GFP is driven by a conserved cis-regulatory element (CR) in the vicinity of neuropeptide coding sequence, either intergenic or intronic, via a basal promoter (pes-10). Panels (B), (C), (D), (E), (F), and (G) show the detection of flp-13, flp-24, and nlp-8 in L1 larva ALA (white arrows) by GFP. The ALA neuron is located dorsal to the pharynx between the anterior and posterior pharyngeal bulbs (arrowheads). Scale bars represent 20 μm.

FIG. 10 depicts gene models of flp-13, flp-24 and nlp-8 with deletion mutations in mutant alleles. Horizontal black bars indicate genomic deletions (www.wormbase.org) and grey blocks represent the exons of coding genes.

FIG. 11 shows that EGF-induced sleep does not involve all ALA-synthesized neuropeptides. Time course of feeding quiescence in wild type was compared with mutants of neuropeptides synthesized in ALA after exposure to heat. Animals lacking flp-5, flp-10, flp-19 and ins-27 are not resistant to sleep induction. egl-3 mutants were used as a general control for deficiency of neuropeptide synthesis.

FIG. 12 shows that FLP-13 peptides (SEQ ID NOS 60-66, respectively, in order of appearance) share a similar structure with zebrafish RFRP-1 (SEQ ID NO: 67) and NPFF (SEQ ID NO: 68) peptides. Alignments of FLP-13 peptides with zebrafish RFRP-1 and NPFF peptides by www.uniprot.org are shown. Hydrophobic amino acids are shaded. Asterisks (*) mark identical amino acids, and dots (. and :) mark similar amino acids.

FIG. 13 shows that FLP-24 peptides (SEQ ID NO: 69) share a similar structure with zebrafish RFRP-1 (SEQ ID NO: 67) and NPFF (SEQ ID NO: 68) peptides. Alignments of FLP-24 peptides with zebrafish RFRP-1 and NPFF peptides by www.uniprot.org are shown. Hydrophobic amino acids are shaded. Asterisks (*) mark identical amino acids, and dots (. and :) mark similar amino acids.

FIG. 14 shows that the conditional activation of zebrafish RFRP-1 suppresses locomotion in C. elegans. Forward (positive centroid velocity) and reverse (negative centroid velocity) movement was recorded in animals recovered from heat shock. The activation of hs:RFRP-1 statistically reduced locomotor activities in wild type animals and the effect is blocked in npr-22 mutants. Basal centroid velocity was normalized by wild type animals without hs:RFRP-1. ***p<0.0001.

FIG. 15 shows that the conditional activation of zebrafish NPFF does not suppress locomotion in C. elegans. Forward (positive centroid velocity) and reverse (negative centroid velocity) movement was recorded in animals recovered from heat shock. The activation of hs:NPFF had no effect on locomotion in wild type animals or npr-22 mutants. Basal centroid velocity was normalized by wild type and npr-22 mutant animals with no hs:NPFF. p>0.05.

FIG. 16 includes four panels, identified as panels (A), (B), (C), and (D). Panel (A) shows a genetic diagram of a conditional overexpression transgene. The RFRP precursor is cleaved enzymatically into three mature RFRP peptides: RFRP-1, RFRP-2, and RFRP-3. The RFRP amino acid shows evolutionary conservation from worms to fish to humans. Panel (B) shows a schematic of the experimental layout for behavioral recording of zebrafish sleep/wake states. Panel (C) shows that the overexpression of RFRP results in a daytime reduction in wake-behavior following heat-shock compared to wild type sibling controls. Panel (D) shows that the overall locomotor daytime activity is reduced following overexpression of RFRP. Data is represented as mean±SEM for 118 WT and 130 hs:RFRP transgenic fish averaged over 3 independent experiments. ***p<0.0001. The sequence alignment shows that the RFRP1, RFRP2, and RFRP3 amino acid sequences (denoted by three horizontal bars) are conserved in vertebrates including the zebrafish (Danio rerio; “NPVF-Dr.pro”) (SEQ ID NO: 74), frog (Xenopus tropicalis; “NPVF-Xt.pro”) (SEQ ID NO: 73), chicken (Gallus gallus; “NPVF-Gg.pro”) (SEQ ID NO: 72), mouse (Mus musculus; “NPVF-Mm.pro”) (SEQ ID NO: 71), and human (Homo sapiens; “NPVF-Hs.pro”) (SEQ ID NO: 70). FIG. 16 also discloses “NPVF_Fr.pro”, “hRFRP1”, “hRFRP2”, and “hRFRP3” as SEQ ID NOS 75-78, respectively.

### DETAILED DESCRIPTION

Neuropeptides play a crucial role in modulating the nervous system through combinatorial inputs with multiple receptors. This modularity of these ligand/receptor systems provides a flexible information-processing and transmission network to translate internal and external signals into behavioral outputs such as feeding, locomotion, pain perception, and mechano-, chemo-, and thermo-sensation.

In vertebrates, numerous neuropeptide/receptor systems have been reported to modulate sleep (see generally Raffa, Peptides 9(4), 915 (1988); Prober et al., J. Neurosci. 26(51), 13400 (2006)), although a universal signaling pathway has not been previously reported. For example, the role of the neuropeptide Y (NPY) system in sleep is elusive and may be state-dependent and species-dependent. The NPY system possesses dual functions in promoting sleep and wakefulness in mammals, depending on the site and vehicle of NPY introduction. In C. elegans, the neuropeptide-like-protein NLP-22 regulates a sleep-like state and the RFamide peptides flp-18 and flp-21 in combination with GPCRs NPR-4/5 and NPR-1 modulate foraging and lethargus, respectively. In comparison, the vertebrate RFamide peptides modulate feeding and reproduction. The instant invention relates to the finding that the ALA-synthesized RFamide peptides can induce sleep via GPCRs, and that homologous vertebrate peptides function similarly. Specifically, agonists of the G-protein coupled receptors (GPCRs) neuropeptide FF receptor 1 (NPFFR1), neuropeptide FF receptor 2 (NPFFR2), neuropeptide Y receptor type 1 (NPY1R), neuropeptide Y receptor type 2 (NPY2R), and tachykinin receptor (TACR) can induce sleep in a subject and antagonists can induce wakefulness.

Previously, it was found that epidermal growth factor (EGF) signaling regulates sleep in a wide range of organisms (see, e.g., Kramer A., et al. Science 294:2511-15 (2001)). In some aspects, the instant invention relates to the finding that the downstream mediators of the EGF sleep pathway are neuromodulators comprising versatile neuropeptide ligand/receptor systems that can promote and maintain sleep by dampening sensory and motor abilities, and yet permit fast reversibility when perturbed by sensory stimulation. Additionally, some aspects of the invention relate to the finding that the functions of these ligand/receptor systems are conserved through evolution from nematodes through vertebrates.

### DEFINITIONS

The articles “a” and “an” are used herein to refer to one or to more than one (i.e., to at least one) of the grammatical object of the article. By way of example, “an element” means one element or more than one element.

The term “about” refers to a range of values of plus or minus 10% of a specified value. For example, the phrase “about 100” includes plus or minus 10% of 100, i.e., from 90 to 110, unless clearly contradicted by context.

Throughout this specification, the word “comprise” or variations such as “comprises” or “comprising” will be understood to imply the inclusion of a stated integer or groups of integers but not the exclusion of any other integer or group of integers.

As used herein, the terms “effective amount” and “therapeutically effective amount” mean a dosage sufficient to produce a desired result, e.g., to induce sleep in a subject, to increase wakefulness in a subject, or to sedate a subject.

The term “prevent” is art-recognized, and when used in relation to a condition, such as sleep, is well understood in the art, and includes administration of a composition which reduces the likelihood of, or delays the onset of, the condition in a subject relative to a subject which does not receive the composition. Thus, prevention of sleep includes, for example, reducing the likelihood that a subject receiving the composition will sleep relative to a subject that does not receive the composition, and/or delaying the onset of sleep, on average, in a treated population versus an untreated control population, e.g., by a statistically and/or clinically significant amount. Similarly, prevention of insomnia includes, for example, increasing the likelihood that a subject receiving the composition will sleep relative to a subject that does not receive the composition, and/or inducing sleep in a treated population versus an untreated control population, e.g., by a statistically and/or clinically significant amount.

The terms “neuropeptide FF receptor 1” and “NPFFR1” refer to vertebrate NPFFR1. In some embodiments, NPFFR1 refers to homologs of human NPFFR1. In some embodiments, NPFFR1 refers to orthologs of human NPFFR1. For example, NPFFR1 may refer to Danio rerio NPFFR-112.

The terms “neuropeptide FF receptor 2” and “NPFFR2” refer to vertebrate NPFFR2. In some embodiments, NPFFR2 refers to homologs of human NPFFR2. In some embodiments, NPFFR2 refers to orthologs of human NPFFR2. For example, NPFFR2 may refer to Danio rerio NPFFR-2.1 or NPFFR-2.2.

The terms “neuropeptide Y receptor 1” and “NPY1R” (“NPYR1”) refer to vertebrate NPY1R. In some embodiments, NPY1R refers to homologs of human NPY1R. In some embodiments, NPY1R refers to orthologs of human NPY1R. For example, NPY1R may refer to Danio rerio NPY1R.

The terms “neuropeptide Y receptor 2” and “NPY2R” (“NPYR2”) refer to vertebrate NPY2R. In some embodiments, NPY2R refers to homologs of human NPY2R. In some embodiments, NPY2R refers to orthologs of human NPY2R. For example, NPY2R may refer to Danio rerio NPY2R.

The terms “tachykinin receptor” and “TACR” refer to vertebrate TACR. In some embodiments, TACR refers to homologs of human TACR. In some embodiments, TACR refers to orthologs of human TACR. For example, TACR may refer to Danio rerio TACR.

As used herein, the terms “treat”, “treating”, and “treatment” include inhibiting the condition, e.g., reducing the onset or symptoms of a condition, disorder, or disease, such as insomnia, fatigue, or narcolepsy. These terms also encompass therapy. Treatment means any manner in which the symptoms of a condition, disorder, or disease are ameliorated or otherwise beneficially altered. Preferably, the subject in need of such treatment is a mammal, more preferably a human.

### Methods for Regulating Sleep in a Subject

In some aspects, the invention relates to a method for regulating sleep in a subject, comprising administering to the subject a composition comprising an agent that modulates the activity of a G-protein coupled receptor (GPCR) selected from neuropeptide FF receptor 1 (NPFFR1), neuropeptide FF receptor 2 (NPFFR2), neuropeptide Y receptor type 1 (NPY1R), neuropeptide Y receptor type 2 (NPY2R), and tachykinin receptor (TACR).

In some embodiments, the subject is human and the GPCR is a human GPCR.

In some embodiments, regulating sleep in the subject comprises inducing sleep in the subject. The invention may relate to a method of sedating the subject. In other embodiments, regulating sleep in the subject comprises increasing wakefulness in a subject. For example, either sleep or wakefulness may be induced in the subject by administering an agonist or antagonist of the GPCR, respectively.

In some embodiments, the invention relates to a method for inducing sleep in a subject, comprising administering to the subject a composition comprising a neuropeptide FF receptor 1 (NPFFR1) agonist. In some embodiments, the invention relates to a method for inducing wakefulness in a subject, comprising administering to the subject a composition comprising a neuropeptide FF receptor 1 (NPFFR1) antagonist.

An agent that modulates the activity of a GPCR may be selected from agonists or antagonists of the GPCR. The agent may be a ligand of the GPCR, e.g., the agent may specifically bind to the GPCR. For example, the agent may bind to an extracellular domain of the GPCR. Similarly, the agent may bind to the transmembrane or cytosolic domain of the GPCR, or the agent may bind to residues in or near the extracellular/transmembrane domains of the GPCR.

An agonist of a GPCR induces a signal transduction pathway mediated by the GPCR upon binding to the GPCR. Accordingly, an agonist modulates the activity of a GPCR by inducing at least one signal transduction pathway mediated by the GPCR, e.g., relative to the level of signaling mediated by the GPCR in the absence of the agonist. For example, an agonist might be a naturally-occurring GPCR ligand or a derivative of a naturally-occurring GPCR ligand, or an agonist may mimic the function of a naturally-occurring GPCR ligand. Similarly, an agonist may induce a conformation of the GPCR that increases the signaling of a GPCR-ligand complex upon binding to the GPCR, e.g., by binding to an allosteric site.

An antagonist of a GPCR inhibits a signal transduction pathway mediated by the GPCR upon binding to the GPCR. Accordingly, an antagonist modulates the activity of a GPCR by inhibiting at least one signal transduction pathway mediated by the GPCR, e.g., relative to the level of signaling mediated by the GPCR in the absence of the antagonist. For example, an antagonist may block a naturally-occurring GPCR ligand from binding to the GPCR, or an antagonist may induce a conformation of the GPCR that reduces the signaling of the GPCR upon binding to the GPCR, e.g., by binding to an allosteric site.

In some embodiments, the GPCR is NPFFR1 or NPFFR2, preferably human NPFFR1 or human NPFFR2. The agent may modulate the activity of more than one GPCR. For example, the agent may modulate the activity of NPFFR2 and either NPY1R and/or NPY2R. The GPCR may be NPFFR1.

In some embodiments, the agent is selected from neuropeptide FF (NPFF), RFamide-related peptide 1 (RFRP1), RFamide-related peptide 2 (RFRP2), or RFamide-related peptide (RFRP3). In some embodiments, the agent is not NPFF, RFRP1, RFRP2, or RFRP3. In some embodiments, the agent is a peptide or a derivative of a peptide. The agent may be a small molecule, e.g., having a molecular weight less than 2000 amu, such as less than 1000 amu, less than 500 amu, less than 400 amu, or less than 300 amu.

In some embodiments, the subject has been diagnosed with a sleep disorder. The subject may have been diagnosed with a psychological condition. The subject may have fatigue, attention deficit hyperactivity disorder, depression, bipolar disorder, schizophrenia, autism, and/or an eating disorder. In some embodiments, the subject has not been diagnosed with a sleep disorder. The subject may or may not have been diagnosed with a psychological condition.

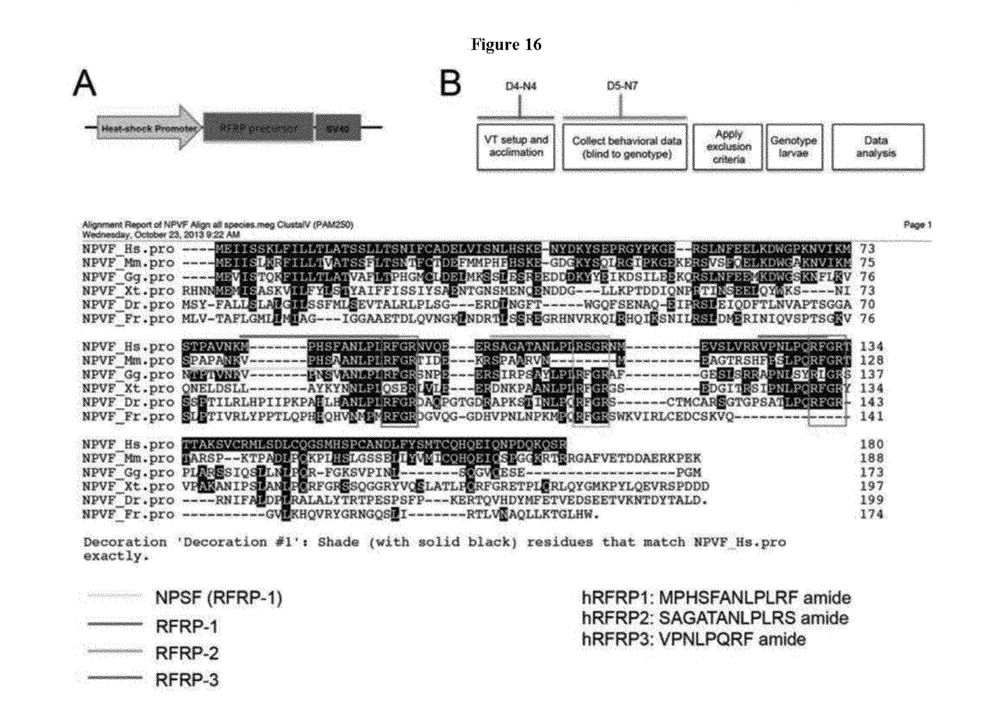

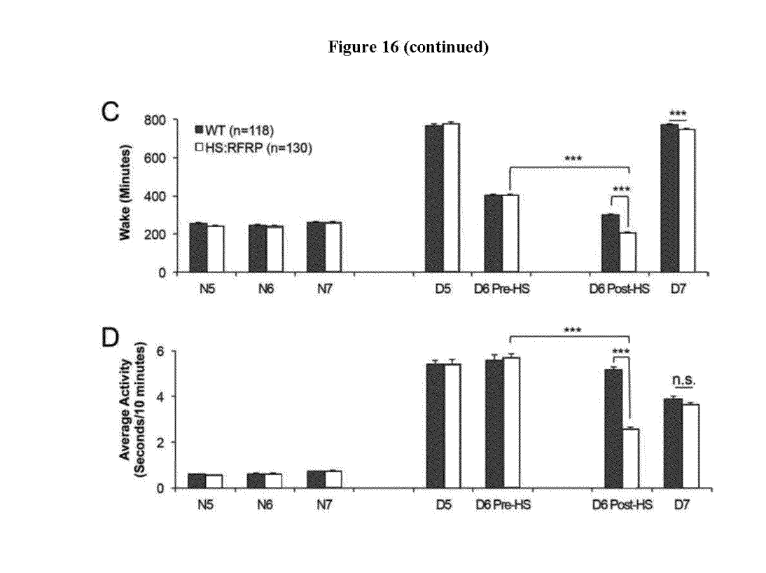

The neuropeptide signaling pathways are conserved throughout vertebrates (FIG. 16). For example, the RFRP1, RFRP2, and RFRP3 neuropeptides occur in fish, amphibians, birds, and mammals. Similarly, the neuropeptide receptor signaling pathways are conserved throughout mammals (FIG. 16; the mouse and human RFRP1 and RFRP2 sequences have 83% and 67% sequence homology, respectively). Thus, the subject may be a vertebrate, such as a fish, frog, bird, or rodent. The subject may be a zebrafish or a mouse. Similarly, the subject may be a mammal, such as a primate, equine, bovine, ovine, porcine, feline, canine, or murine. In some embodiments, the subject is a human. In some embodiments, the invention relates to methods of sedating a large animal, e.g., to transport the animal, for a research experiment, or to perform a medical procedure on the animal.

Administration may comprise, for example, oral administration. Accordingly, the composition may be formulated for oral administration.

### Methods for Characterizing a Sleep Disorder

In certain aspects, the invention relates to a method of characterizing a sleep disorder in a subject, the method comprising: obtaining a sample of a nucleic acid from the subject, wherein the nucleic acid encodes a NPFFR1, NPFFR2, NPY1R, NPY2R, TACR, NPFF, RFRP1, RFRP2, and/or RFRP3 gene; sequencing the nucleic acid; comparing the nucleotide sequence encoded by the nucleic acid with a reference nucleotide sequence from a subject that does not have a sleep disorder; and diagnosing the subject with a sleep disorder if the nucleotide sequence is different from the reference nucleotide sequence. In certain aspects, the invention relates to a method of characterizing a sleep disorder in a subject, the method comprising: obtaining a sample of a nucleic acid from the subject, wherein the nucleic acid encodes a NPFFR1, NPFFR2, NPY1R, NPY2R, TACR, NPFF, RFRP1, RFRP2, and/or RFRP3 gene; sequencing the nucleic acid; comparing the amino acid sequence encoded by the nucleic acid with a reference amino acid sequence from a subject that does not have a sleep disorder; and diagnosing the subject with a sleep disorder (or even a particular sleep disorder) if the amino acid sequence is different from the reference amino acid sequence. Preferably, the subject is human and the nucleic acid encodes a human gene.

The sample may be obtained directly from the subject or indirectly, e.g., from a physician or an archive. The subject may be diagnosed with a sleep disorder, for example, if the nucleic acid encodes a nucleotide or amino acid sequence for NPFFR1, NPFFR2, NPY1R, NPY2R, and/or TACR that prevents the expression of a full-length protein (e.g., the sequence comprises a truncation mutation), prevents the proper folding of the protein, prevents the protein from localizing to the extracellular membrane, prevents binding to a ligand, prevents the binding of a ligand from initiating a signaling pathway, prevents an interaction with one or more signaling molecules, decreases the stability of the protein, and/or targets the protein for degradation. The subject may be diagnosed with a sleep disorder if the nucleotide sequence comprises a mutation in a promoter or enhancer region that affects the transcription of the protein, e.g., by increasing or decreasing the rate at which a gene encoding the protein is transcribed. The subject may be diagnosed with a sleep disorder, for example, if the nucleic acid encodes an amino acid sequence for NPFF, RFRP1, RFRP2, and/or RFRP3 that prevents the expression of a full-length peptide (e.g., the sequence comprises a truncation mutation), prevents the post-translational modification of the peptide (e.g., prevents the cleavage of a precursor protein or peptide into RFRP1, RFRP2, and/or RFRP3), prevents cells from secreting the peptide, decreases the half-life of the peptide, and/or alters the binding affinity of the peptide for one or more GPCRs.

Methods for Identifying Agonists and Antagonists of NPFFR1, NPFFR2, NPY1R, NPY2R, and/or TACR

In some aspects, the invention relates to a method of identifying whether a compound affects a condition in a subject, comprising: administering the compound to a test subject and monitoring the activity of the test subject, wherein the test subject comprises a genetic modification that reduces the expression of NPFFR1, NPFFR2, NPY1R, NPY2R, and/or TACR, and/or a homolog or ortholog of any one of NPFFR1, NPFFR2, NPY1R, NPY2R and/or TACR; administering the compound to a control subject and monitoring the activity of the control subject, wherein the control subject does not comprise the genetic modification; comparing the activity of the control subject and the test subject; and identifying whether the compound affects the condition, wherein the condition is selected from fatigue, sleep, wakefulness, attention deficit hyperactivity disorder, chronic fatigue syndrome, depression, bipolar disorder, schizophrenia, autism, and an eating disorder. Identifying whether the compound affects the condition may comprise determining whether the activity of the test subject is different from the activity of the control subject, e.g., whether the test subject sleeps more or less than the control subject or whether the test subject is more or less active than the control subject. For example, the activity may be sleep and monitoring the activity may comprise measuring duration of sleep. The condition may be sleep or wakefulness, e.g., the method may comprise identifying whether a compound affects sleep or wakefulness in the subject.

The subject may be a vertebrate such as a zebrafish or mouse. The genetic modification may be a knockout or knockdown mutation of a gene encoding NPFFR1, NPFFR2, NPY1R, NPY2R, and/or TACR, and/or a homolog or ortholog of any one of NPFFR1, NPFFR2, NPY1R, NPY2R and/or TACR.

In certain aspects, the invention relates to a method of identifying whether a compound affects a condition in a subject, comprising: administering the compound to a test subject and monitoring the activity of the test subject, wherein the test subject is Caenorhabditis elegans comprising a genetic modification that reduces the expression of neuropeptide receptor 7 (NPR-7) or neuropeptide receptor 22 (NPR-22); administering the compound to a control subject and monitoring the activity of the control subject, wherein the control subject is Caenorhabditis elegans and the control subject does not comprise the genetic modification; comparing the activity of the control subject and the test subject; and identifying whether the compound affects the condition. Identifying whether the compound affects the condition may comprise determining whether the activity of the test subject is different from the activity of the control subject. The activity may be, for example, feeding quiescence, locomotion quiescence, pumping, or response latency. The condition may be sleep or wakefulness.

### Agents

Neuropeptide receptor ligands are well known in the art (see generally Mankus, J. V. & C. F. McCurdy, Future Med Chem 4(9):1085-92 (2012)). For example, the agent may be RF9 (Formula V; 2-Adamantanecarbonyl-Arg-Phe-NH₂) or GR231118 (CAS 158859-98-4). RF9 is an antagonist of NPFFRs, e.g., RF9 is a NPFFR1 antagonist and a NPFFR2 antagonist, and RF9 also specifically binds to other GPCRs, e.g., NPY1R. GR231118 is a NPY1R antagonist, and GR231118 also specifically binds to other GPCRs, e.g., NPY4R. The agent may be BIBP-3226 (Formula VI; N-[(1R)]-4-[(Aminoiminomethyl)amino-1-[[[(4-hydroxyphenyl)methyl]amino]carbonyl]butyl-α-phenylbenzeneacetamide trifluoroacetate), which is an antagonist of NPY1R and NPFFRs. The agent may be dNPA (d-Asn-Pro-(NHMe)Ala-Phe-Leu-Phe-Gln-Pro-Gln-Arg-Phe-NH₂), which is a NPFFR2 agonist. Other neuropeptide receptor ligands are well known in the art (see, e.g., U.S. Pat. No. 7,727,979; U.S. Pat. No. 7,544,691; U.S. Pat. No. 6,960,574; U.S. Patent Application Publication No. 2007/0123510; U.S. Patent Application Publication No. 2005/0136444; U.S. Patent Application Publication No. 2003/0176314; U.S. Patent Application Publication No. 2003/0139431; PCT Patent Application Publication No. WO 2009/038012; PCT Patent Application Publication No. WO 2004/080965; PCT Patent Application Publication No. WO 2003/026657, hereby incorporated by reference, especially for the compounds disclosed therein).

In some embodiments, the agent comprises a guanidino group. For example, the agent may comprise an arginine moiety. In some embodiments, the agent does not comprise a guanidino group. The agent may comprise a phenylalanine moiety and/or an adamantane moiety.

The agent may be selected from N-(4,6-dimethyl-2-quinolinyl)guanidine and N-(4,7-dimethyl-2-quinolinyl)guanidine, which are antagonists of NPFFR1 and agonists of NPFFR2 (U.S. Patent Application Publication No. 2003/0139431, hereby incorporated by reference, especially for the compounds disclosed therein). The agent may be selected from N-(6-benzyl-4-methyl-2-quinazolinyl)guanidine, N-(6-butyl-4-methyl-2-quinazolinyl)guanidine, N-(6-hexyl-4-methyl-2-quinazolinyl)guanidine, N-(4-methyl-6-pentyl-2-quinolinyl)guanidine, and N-(6-butyl-4-methyl-2-quinolinyl)guanidine, which are agonists of both NPFFR1 and NPFFR2 (U.S. Patent Application Publication No. 2003/0139431, hereby incorporated by reference, especially for the compounds disclosed therein). The compound may be selected from N-(4-methyl-2-quinolinyl)guanidine, N-(4-ethyl-7-methyl-2-quinolinyl)guanidine, and N-(4,8-dimethyl-2-quinolinyl)guanidine, which are antagonists of both NPFFR1 and NPFFR2 (U.S. Patent Application Publication No. 2003/0139431, hereby incorporated by reference, especially for the compounds disclosed therein).

The agent may be selected from N-(5-ethyl-5-methyl-4,5,6,7-tetrahydro-benzothiazole-2-yl)-guanidine, N-(4-tert-butyl-4,5,6,7-tetrahydro-benzothiazole-2-yl)-guanidine, N-(6-isopropyl-4,5,6,7-tetrahydro-benzothiazole-2-yl)-guanidine, N-(5-butyl-5,6,7,8-tetrahydro-4H-cycloheptathiazol-2-yl)-guanidine, N-(4-ethyl-4-methyl-4,5,6,7-tetrahydro-benzothiazole-2-yl)-guanidine, N-[6-(3,4-dimethoxyphenyl)-4,5,6,7-tetrahydro-benzothiazole-2-yl]-guanidine, N-(5-butyl-4,5,6,7-tetrahydro-benzothiazole-2-yl)-guanidine, N-(6-phenyl-4,5,6,7-tetrahydro-benzothiazole-2-yl)-guanidine, N-(6-propyl-4,5,6,7-tetrahydro-benzothiazole-2-yl)-guanidine, N-(4-cyclohex-1-enyl-4,5,6,7-tetrahydro-benzothiazole-2-yl)-guanidine, N-(4-sec-butyl-4,5,6,7-tetrahydro-benzothiazole-2-yl)-guanidine, N-(4-isobutyl-4-methyl-4,5,6,7-tetrahydro-benzothiazole-2-yl)-guanidine, N-(6-tert-butyl-4,5,6,7-tetrahydro-benzothiazole-2-yl)-guanidine, N-[6-(1,1-dimethyl-propyl)-4,5,6,7-tetrahydro-benzothiazole-2-yl]-guanidine, N-[6-(3-methoxy-phenyl)-4,5,6,7-tetrahydro-benzothiazole-2-yl]-guanidine, N-(6-thiophene-2-yl-4,5,6,7-tetrahydro-benzothiazole-2-yl)-guanidine, N-[6-(4-fluorophenyl)-4,5,6,7-tetrahydro-benzothiazole-2-yl]-guanidine, N-(4-allyl-4,5,6,7-tetrahydro-benzothiazole-2-yl)-guanidine, N-[6-(3-fluorophenyl)-4,5,6,7-tetrahydro-benzothiazole-2-yl]-guanidine, N-(6-cyano-6-phenyl-4,5,6,7-tetrahydro-benzothiazole-2-yl)-guanidine, N-(4-phenyl-4,5,6,7-tetrahydro-benzothiazole-2-yl)-guanidine, N-(6,6-diphenyl-4,5,6,7-tetrahydro-benzothiazole-2-yl)-guanidine, N-[6-(4-methoxy-phenyl)-4,5,6,7-tetrahydro-benzothiazole-2-yl]-guanidine, N-(5-phenyl-5,6,7,8-tetrahydro-4H-cycloheptathiazol-2-yl)-guanidine, N-(6-benzo[1,3]dioxol-5-yl-4,5,6,7-tetrahydro-benzothiazole-2-yl)-guanidine, N-[6-(4-cyanophenyl)-4,5,6,7-tetrahydro-benzothiazole-2-yl]-guanidine, N-(4-benzyl-4,5,6,7-tetrahydro-benzothiazole-2-yl)-guanidine, N-(5-methyl-5-phenyl-4,5,6,7-tetrahydro-benzothiazole-2-yl)-guanidine, N-[6-(3,5-bis-trifluoromethylphenyl)-4,5,6,7-tetrahydro-benzothiazole-2-yl]-guanidine, N-(6-o-tolyl-4,5,6,7-tetrahydro-benzothiazole-2-yl)-guanidine, N-(6-m-tolyl-4,5,6,7-tetrahydro-benzothiazole-2-yl)-guanidine, N-[6-(2-ethyl-phenyl)-4,5,6,7-tetrahydro-benzothiazole-2-yl]-guanidine, N-[6-(4-chlorophenyl)-4,5,6,7-tetrahydro-benzothiazole-2-yl]-guanidine, N-[6-(4-benzyloxy-phenyl)-4,5,6,7-tetrahydro-benzothiazole-2-yl]-guanidine, N-(6-phenyl-4,5,6,7-tetrahydro-benzothiazole-2-yl-4-spiro-cyclohexane)-guanidine, N-(6-p-tolyl-4,5,6,7-tetrahydro-benzothiazole-2-yl)-guanidine, N-(5,5-dimethyl-4,5,6,7-tetrahydro-benzothiazole-2-yl)-guanidine, N-(6,6-dimethyl-4,5,6,7-tetrahydro-benzothiazole-2-yl)-guanidine, N-(5-methyl-4,5,6,7-tetrahydro-benzothiazole-2-yl)-guanidine, N-(4-methyl-4-propyl-4,5,6,7-tetrahydro-benzothiazole-2-yl)-guanidine, N-(7-methyl-4,5,6,7-tetrahydro-benzothiazole-2-yl)-guanidine, N-(4,4-dimethyl-4,5,6,7-tetrahydro-benzothiazole-2-yl)-guanidine, N-(4-methyl-4,5,6,7-tetrahydro-benzothiazole-2-yl)-guanidine, and N-(6-methyl-4,5,6,7-tetrahydro-benzothiazole-2-yl)-guanidine (U.S. Pat. No. 7,727,979, hereby incorporated by reference, especially for the compounds disclosed therein).

The agent may be selected from 2-guanidino-6,7-dihydro-4H-thiazolo[5,4-c]pyridine-5-carboxylic acid tert-butyl ester, N-(5-hexyl-4,5,6,7-tetrahydro-thiazolo[5,4-c]pyridine-2-yl)-guanidine, N-[5-(2-cyclohexyl-ethyl)-4,5,6,7-tetrahydro-thiazolo[5,4-c]pyridine-2-yl]-guanidine, N-(5-ethyl-4,5,6,7-tetrahydro-thiazolo[5,4-c]pyridine-2-yl)-guanidine, 2-guanidino-6,7-dihydro-4H-thiazolo[5,4-c]pyridine-5-carboxylic acid butyl ester, N-[5-(propane-2-sulphonyl)-4,5,6,7-tetrahydro-thiazolo[5,4-c]pyridine-2-yl]-guanidine, N-(5-phenylacetyl-4,5,6,7-tetrahydro-thiazolo[5,4-c]pyridine-2-yl)-guanidine, 2-guanidino-6,7-dihydro-4H-thiazolo[5,4-c]pyridine-5-carboxylic acid benzyl ester, N-(5-pentanoyl-4,5,6,7-tetrahydro-thiazolo[5,4-c]pyridine-2-yl)-guanidine, 2-guanidino-6,7-dihydro-4H-thiazolo[5,4-c]pyridine-5-thiocarboxylic acid propyl amide, N-[5-(2-propyl-pentanoyl)-4,5,6,7-tetrahydro-thiazolo[5,4-c]pyridine-2-yl]-guanidine, N-(5-benzyl-4,5,6,7-tetrahydro-thiazolo[5,4-c]pyridine-2-yl)-guanidine, N-(5-prop-2-ynyl-4,5,6,7-tetrahydro-thiazolo[5,4-c]pyridine-2-yl)-guanidine, N-(5-cyclopropanecarbonyl-4,5,6,7-tetrahydro-thiazolo[5,4-c]pyridine-2-yl)-guanidine, N-[5-(butane-1-sulphonyl)-4,5,6,7-tetrahydro-thiazolo[5,4-c]pyridine-2-yl]-guanidine, N-(5-isobutyryl-4,5,6,7-tetrahydro-thiazolo[5,4-c]pyridine-2-yl)-guanidine, N-[5-(2,2-dimethyl-propionyl)-4,5,6,7-tetrahydro-thiazolo[5,4-c]pyridine-2-yl]-guanidine, 2-guanidino-6,7-dihydro-4H-thiazolo[5,4-c]pyridine-5-thiocarboxylic acid benzyl amide, 2-guanidino-6,7-dihydro-4H-thiazolo[5,4-c]pyridine-5-carboxylic acid tert-butyl amide, N-(5-but-3-enoyl-4,5,6,7-tetrahydro-thiazolo[5,4-c]pyridine-2-yl)-guanidine, N-(5-benzyl-5,6,7,8-tetrahydro-4H-thiazolo[4,5-c]azepine-2-yl)-guanidine, 3-(2-guanidino-6,7-dihydro-4H-thiazolo[5,4-c]pyridine-5-yl)-propionic acid ethyl ester, 2-guanidino-6,7-dihydro-4H-thiazolo[5,4-c]pyridine-5-carboxylic acid pentyl amide, N-[5-(2-methoxy-acetyl)-4,5,6,7-tetrahydro-thiazolo[5,4-c]pyridine-2-yl]-guanidine, N-(5-cyclopropylmethyl-4,5,6,7-tetrahydro-thiazolo[5,4-c]pyridine-2-yl)-guanidine, N-(5-methanesulphonyl-4,5,6,7-tetrahydro-thiazolo[5,4-c]pyridine-2-yl)-guanidine, N-[5-(3-methyl-butyryl)-4,5,6,7-tetrahydro-thiazolo[5,4-c]pyridine-2-yl]-guanidine, 2-guanidino-6,7-dihydro-4H-thiazolo[5,4-c]pyridine-5-thiocarboxylic acid-(2-methoxy-1-methyl-ethyl)-amide, 2-guanidino-6,7-dihydro-4H-thiazolo[5,4-c]pyridine-5-carboxylic acid phenyl amide, [3-(2-guanidino-6,7-dihydro-4H-thiazolo[5,4-c]pyridine-5-yl)-3-oxo-propyl]-carbamic acid tert-butyl ester, N-[5-(4-dimethylamino-butyryl)-4,5,6,7-tetrahydro-thiazolo[5,4-c]pyridine-2-yl]-guanidine, N-(5-propyl-4,5,6,7-tetrahydro-thiazolo[5,4-c]pyridine-2-yl)-guanidine, 2-guanidino-6,7-dihydro-4H-thiazolo[5,4-c]pyridine-5-thiocarboxylic acid isopropyl amide, N-(4,5,6,7-tetrahydro-benzothiazole-2-yl)-guanidine, (2-guanidino-4,5,6,7-tetrahydro-benzothiazole-4-yl)-ethyl acetate ethyl ester, N-(4-hydroxymethyl-4,5,6,7-tetrahydro-benzothiazole-2-yl)-guanidine, N-(4-tosyloxymethyl-4,5,6,7-tetrahydro-benzothiazole-2-yl)-guanidine, N-(4-azidomethyl-4,5,6,7-tetrahydro-benzothiazole-2-yl)-guanidine, N-(4-aminomethyl-4,5,6,7-tetrahydro-benzothiazole-2-yl)-guanidine, N-(6-acetylaminomethyl-4,5,6,7-tetrahydro-benzothiazole-2-yl)-guanidine, N-(5-ethyl-5-methyl-4,5,6,7-tetrahydro-benzothiazole-2-yl)-guanidine, N-(5,5-dimethyl-4,5,6,7-tetrahydro-benzothiazole-2-yl)-guanidine, N-(5,5-dimethyl-6-phenyl-4,5,6,7-tetrahydro-benzothiazole-2-yl)-guanidine, N-(4-tert-butyl-4,5,6,7-tetrahydro-benzothiazole-2-yl)-guanidine, N-(6-isopropyl-4,5,6,7-tetrahydro-benzothiazole-2-yl)-guanidine, N-(5,5,7-trimethyl-4,5,6,7-tetrahydro-benzothiazole-2-yl)-guanidine, N-(6,6-dimethyl-4,5,6,7-tetrahydro-benzothiazole-2-yl)-guanidine, N-(5-butyl-5,6,7,8-tetrahydro-4H-cycloheptathiazol-2-yl)-guanidine, N-(4-ethyl-4-methyl-4,5,6,7-tetrahydro-benzothiazole-2-yl)-guanidine, N-[6-(3,4-dimethoxyphenyl)-4,5,6,7-tetrahydro-benzothiazole-2-yl]-guanidine, N-(5-butyl-4,5,6,7-tetrahydro-benzothiazole-2-yl)-guanidine, N-(6-phenyl-4,5,6,7-tetrahydro-benzothiazole-2-yl)-guanidine, N-(5-methyl-4,5,6,7-tetrahydro-benzothiazole-2-yl)-guanidine, N-(4-methyl-4-propyl-4,5,6,7-tetrahydro-benzothiazole-2-yl)-guanidine, N-(6-propyl-4,5,6,7-tetrahydro-benzothiazole-2-yl)-guanidine, N-(4-cyclohex-1-enyl-4,5,6,7-tetrahydro-benzothiazole-2-yl)-guanidine, N-(4-sec-butyl-4,5,6,7-tetrahydro-benzothiazole-2-yl)-guanidine, N-(4-isobutyl-4-methyl-4,5,6,7-tetrahydro-benzothiazole-2-yl)-guanidine N-(6-tert-butyl-4,5,6,7-tetrahydro-benzothiazole-2-yl)-guanidine, 2-guanidino-6-phenyl-4,5,6,7-tetrahydro-benzothiazole-6-carboxylic acid ethyl ester, N-[6-(1,1-dimethyl-propyl)-4,5,6,7-tetrahydro-benzothiazole-2-yl]-guanidine, N-(7-methyl-4,5,6,7-tetrahydro-benzothiazole-2-yl)-guanidine, N-[6-(3-methoxy-phenyl)-4,5,6,7-tetrahydro-benzothiazole-2-yl]-guanidine, N-(6-thiophene-2-yl-4,5,6,7-tetrahydro-benzothiazole-2-yl)-guanidine, N-(5,5,7,7-tetramethyl-4,5,6,7-tetrahydro-benzothiazole-2-yl)-guanidine, N-[6-(4-fluorophenyl)-4,5,6,7-tetrahydro-benzothiazole-2-yl]-guanidine 2-guanidino-4,5,6,7-tetrahydro-benzothiazole-6-carboxylic acid ethyl ester N-(4,4-dimethyl-4,5,6,7-tetrahydro-benzothiazole-2-yl)-guanidine, N-(4-methyl-4,5,6,7-tetrahydro-benzothiazole-2-yl)-guanidine, N-(4,5,6,7-tetrahydro-benzothiazole-2-yl-4-spiro-cyclohexane)-guanidine, N-(5,6,7,8-tetrahydro-4H-cycloheptathiazol-2-yl)-guanidine, N-(4-allyl-4,5,6,7-tetrahydro-benzothiazole-2-yl)-guanidine, N-(6-methyl-4,5,6,7-tetrahydro-benzothiazole-2-yl)-guanidine, N-[6-(3-fluorophenyl)-4,5,6,7-tetrahydro-benzothiazole-2-yl]-guanidine, N-(6-cyano-6-phenyl-4,5,6,7-tetrahydro-benzothiazole-2-yl)-guanidine N-(4-phenyl-4,5,6,7-tetrahydro-benzothiazole-2-yl)-guanidine N-(6,6-diphenyl-4,5,6,7-tetrahydro-benzothiazole-2-yl)-guanidine N-[6-(4-methoxy-phenyl)-4,5,6,7-tetrahydro-benzothiazole-2-yl]-guanidine, N-(5-phenyl-5,6,7,8-tetrahydro-4H-cycloheptathiazol-2-yl)-guanidine N-(6,7-dihydro-4H-pyrano[4,3-d]thiazol-2-yl)-guanidine, N-(6-benzo[1,3]dioxol-5-yl-4,5,6,7-tetrahydro-benzothiazole-2-yl)-guanidine, 2-guanidino-4,5,6,7-tetrahydro-benzothiazole-6-carboxylic acid propyl amide, N-[6-(4-cyanophenyl)-4,5,6,7-tetrahydro-benzothiazole-2-yl]-guanidine, N-(4-benzyl-4,5,6,7-tetrahydro-benzothiazole-2-yl)-guanidine, N-(5-methyl-5-phenyl-4,5,6,7-tetrahydro-benzothiazole-2-yl)-guanidine, N-[6-(3,5-to-trifluoromethylphenyl)-4,5,6,7-tetrahydro-benzothiazole-2-yl]-guanidine, N-(6-o-tolyl-4,5,6,7-tetrahydro-benzothiazole-2-yl)-guanidine, N-(6-m-tolyl-4,5,6,7-tetrahydro-benzothiazole-2-yl)-guanidine, N-[6-(2-ethyl-phenyl)-4,5,6,7-tetrahydro-benzothiazole-2-yl]-guanidine, N-[6-(4-chlorophenyl)-4,5,6,7-tetrahydro-benzothiazole-2-yl]-guanidine, 2-guanidino-4,5,6,7-tetrahydro-benzothiazole-4-carboxylic acid benzyl amide, N-(5,6-dihydro-4H-cyclopentathiazol-2-yl)-guanidine, N-[6-(4-benzyloxy-phenyl)-4,5,6,7-tetrahydro-benzothiazole-2-yl]-guanidine, 2-guanidino-4,5,6,7-tetrahydro-benzothiazole-4-carboxylic acid methyl phenethyl amide, N-(6-phenyl-4,5,6,7-tetrahydro-benzothiazole-2-yl-4-spiro-cyclohexane)-guanidine, N-(6-p-tolyl-4,5,6,7-tetrahydro-benzothiazole-2-yl)-guanidine, 2-guanidino-4,5,6,7-tetrahydro-benzothiazole-4-carboxylic acid-(3-methyl-butyl)-amide, N-(4-tert-butyl-6-phenyl-4,5,6,7-tetrahydro-benzothiazole-2-yl)-guanidine 2-guanidino-4,5,6,7-tetrahydro-benzothiazole-6-carboxylic acid phenyl amide, 2-guanidino-4,5,6,7-tetrahydro-benzothiazole-4-carboxylic acid butyl ethyl amide, N-[4-(2-cyano-ethyl)-4,5,6,7-tetrahydro-benzothiazole-2-yl]-guanidine, 2-guanidino-4,5,6,7-tetrahydro-benzothiazole-4-carboxylic acid ethyl ester, 2-guanidino-4,5,6,7-tetrahydro-benzothiazole-4-carboxylic acid dipropyl amide, 2-guanidino-4,5,6,7-tetrahydro-benzothiazole-4-carboxylic acid phenyl amide, 2-guanidino-4,5,6,7-tetrahydro-benzothiazole-6-carboxylic acid allyl amide, 2-guanidino-4,5,6,7-tetrahydro-benzothiazole-4-carboxylic acid propyl amide, N-[4-(piperidine-1-carbonyl)-4,5,6,7-tetrahydro-benzothiazole-2-yl]-guanidine, 2-guanidino-4,5,6,7-tetrahydro-benzothiazole-4-carboxylic acid allyl amide, 2-guanidino-4,5,6,7-tetrahydro-benzothiazole-6-carboxylic acid-(3-methyl-butyl)-amide, N-[4-(morpholine-4-carbonyl)-4,5,6,7-tetrahydro-benzothiazole-2-yl]-guanidine, and 2-guanidino-4,5,6,7-tetrahydro-benzothiazole-4-carboxylic acid diisopropyl amide (U.S. Pat. No. 7,727,979, hereby incorporated by reference, especially for the compounds disclosed therein).

The agent may be selected from 1-(4-Fluorobenzylideneamino)guanidine, 1-[3-(Trifluoromethyl)benzylideneamino]guanidine, 1-[1-(3-Bromophenyl)ethylideneamino]guanidine, 1-(5-Fluoro-2-nitrobenzylideneamino)guanidine, 1-[(Benzo[1,3]dioxol-5-yl methylideneamino]guanidine, 1-[(Anthracen-9-yl)methylideneamino]guanidine, 1-(3,5-Dimethoxybenzylideneamino)guanidine, 1-(2,4-Dichlorobenzylideneamino)guanidine, 1-(3-Fluoro-4-methoxybenzylideneamino)guanidine, 1-(3-Bromo-4-fluorobenzylideneamino)guanidine, 1-(3,4,5-Trimethoxybenzylideneamino)guanidine, 1-(4-Fluoro-3-methylbenzylideneamino)guanidine, 1-(3-Chloro-4-fluorobenzylideneamino)guanidine, 1-(3-Bromo-4-methoxybenzylideneamino)guanidine, 1-(2,5-Difluorobenzylideneamino)guanidine, 1-(2,4-Difluorobenzylideneamino)guanidine, 1-(2,3-Dichlorobenzylideneamino)guanidine, 1-(4-Bromo-2-fluorobenzylideneamino)guanidine, 1-(4-Phenylbenzylideneamino)guanidine, 1-(4-Phenoxybenzylideneamino)guanidine, 1-(3-Phenoxybenzylideneamino)guanidine, 1-(3,5-Di-tert-butyl-2-hydroxybenzylideneamino)guanidine, 1-(2,3,5-Trichlorobenzylideneamino)guanidine, 1-(3,5-Dibromo-4-hydroxybenzylideneamino)guanidine, 1-(4-Isopropoxybenzylideneamino)guanidine, 1-(3,4-Diethoxybenzylideneamino)guanidine, 1-(3,5-Difluorobenzylideneamino)guanidine, 1-(3,4-Dibromobenzylideneamino)guanidine, 1-(3,4-Dibromobenzylideneamino)guanidine, 1-(4-Chloro-3-fluorobenzylideneamino)guanidine, 1-(3-Chloro-4-hydroxybenzylideneamino)guanidine, 1-(4-Fluoro-3-nitrobenzylideneamino)guanidine, 1-(3,5-Dimethyl-4-hydroxybenzylideneamino)guanidine, 1-(4-Methoxy-2,3-dimethylbenzylideneamino)guanidine, 1-[4-Chloro-3-(trifluoromethyl)benzylideneamino]guanidine, 1-(3-Bromo-4,5-dimethoxybenzylideneamino)guanidine, 1-[3,4-Dihydro-2H-benzo[b][1,4]dioxepin-7-yl)methylideneamino]guanidine, [(Cyclohexylphenylmethylideneamino]guanidine, 1-[1-(2,3-Dihydro-benzo[1,4]dioxin-6-yl)ethylideneamino]guanidine, 1-(4-Benzyloxy-3-chlorobenzylideneamino)guanidine, 1-(4-Allyloxy-3-chlorobenzylideneamino)guanidine, 1-(3-Chloro-4-methoxybenzylideneamino)guanidine, 1-[3-Chloro-4-(4-cyanobutoxyl)benzylideneamino]guanidine, 1-[3-Chloro-4-(3-phenoxypropoxyl)benzylideneamino]guanidine, 1-[3-Chloro-4-(2-phenylethoxyl)benzylideneamino]guanidine, 1-(3-Chloro-4-hexyloxybenzylideneamino)guanidine, 1-(3-Chloro-4-propoxyobenzylideneamino)guanidine, 1-[3-Chloro-4-(2-methylpropoxyl)benzylideneamino]guanidine, 1-[3-Chloro-4-(4-methylpentoxyl)benzylideneamino]guanidine, 1-[3-Chloro-4-(4-cyclohexylmethoxyl)benzylideneamino])guanidine, 1-[3-Chloro-4-(2-ethylbutoxyl)benzylideneamino]guanidine, 1-(3-Chloro-4-octyloxybenzylideneamino)guanidine, 1-[3-Chloro-4-(2-ethoxy-ethoxy)benzylideneamino])guanidine, 1-(2-Phenylbenzylideneamino)guanidine, 1-(3,4-Dichlorophenyl)-1-(propylideneaminoguanidine), 1-[4-(2-Fluorophenyl)benzylideneamino]guanidine, 1-[3-(2-Trifluoromethylphenyl)benzylideneamino]guanidine, 1-(5-Chloro-2,3-dimethoxybenzylideneamino)guanidine, 1-[2-Fluoro-4-(trifluoromethyl)benzylideneamino]guanidine, 1-[2,4-Bis(trifluoromethyl)benzylideneamino]guanidine, 1-[2,3-Difluoro-4-(trifluoromethyl)benzylideneamino]guanidine, 1-[3-Fluoro-4-(trifluoromethyl)benzylideneamino]guanidine, 1-[3-Nitro-4-(trifluoromethyl)benzylideneamino]guanidine, 1-[2-Fluoro-3-(trifluoromethyl)benzylideneamino]guanidine, 1-[2-Fluoro-5-(trifluoromethyl)benzylideneamino]guanidine, 1-[3-Fluoro-5-(trifluoromethyl)benzylideneamino]guanidine, 1-[4-Fluoro-3-(trifluoromethyl)benzylideneamino]guanidine, 1-[2-Chloro-5-(trifluoromethyl)benzylideneamino]guanidine, 1-[2-Chloro-3-(trifluoromethyl)benzylideneamino]guanidine, 1-[3-Chloro-2-fluoro-5-(trifluoromethyl)benzylideneamino]guanidine, 1-[(4-Fluoro-1-naphthalen-1-yl)methylideneamino]guanidine, 1-[4-Methoxy-3-(trifluoromethyl)benzylideneamino]guanidine, 1-[2-Methoxy-5-(trifluoromethyl)benzylideneamino]guanidine, 1-[Naphthalen-2-yl-methylideneamino]guanidine, 1-[5-Bromo-2-ethoxybenzylideneamino]guanidine, 1-[2,4-Dimethylbenzylideneamino]guanidine, 1-[4-Chloro-3-nitrobenzylideneamino]guanidine, 1-(4-Benzyloxy-2-hydroxybenzylideneamino)guanidine, 1-[(1H-Indol-5-yl)methylideneamino]guanidine, 1-(4-Butoxybenzylideneamino)guanidine, 1-(4-Cyanobenzylideneamino)guanidine, 1-(2,5-Dimethoxybenzylideneamino)guanidine, 1-(2-Benzyloxy-3-methoxybenzylideneamino)guanidine, 1-[1-(2-Methoxy-naphthalen-1-yl)methylideneamino]guanidine, 1-(4-Hydroxy-3-methoxy-5-nitrobenzylideneamino)guanidine, 1-(3,4-Dihydroxybenzylideneamino)guanidine, 1-(3-Bromobenzylideneamino)guanidine, 1-(3,5-Dibromobenzylideneamino)guanidine, 1-[1-(3,4-Dichlorophenyl)ethylideneamino]guanidine, 1-(4-n-Hexyloxybenzylideneamino)guanidine, 1-(3,4-Dibenzyloxybenzylideneamino)guanidine, 1-[(6-Bromobenzo[1,3]dioxol-5-yl)methylideneamino]guanidine, 1-[1-(4-Bromophenyl)ethylideneamino]guanidine, 1-[1-(3-Methylphenyl)ethylideneamino]guanidine, 1-(3-Methylbenzylideneamino)guanidine, 1-(3,4-Dimethylbenzylideneamino)guanidine, 1-[1-(4-Ethylphenyl)ethylideneamino]guanidine, 1-[1-(3,4-Dimethylphenyl)ethylideneamino]guanidine, 1-(4-n-pentylbenzylideneamino)guanidine, 1-[1-(4-n-Heptylphenyl)ethylideneamino]guanidine, 1-[1-(5,6,7,8-Tetrahydronaphthalen-2-yl)ethylideneamino]guanidine, 1-(4-Ethylbenzylideneamino)guanidine, 1-[1-(2-Bromophenyl)ethylideneamino]guanidine, 1-{1-[3-(Trifluoromethyl)phenyl]ethylideneamino}guanidine, 1-{1-[3,5-Bis-(trifluoromethyl)phenyl]ethylideneamino}guanidine, 1-[1-(2,5-Dimethoxyphenyl)ethylideneamino]guanidine, 1-[1-(2-Hydroxy-4-methoxyphenyl)ethylideneamino]guanidine, 1-[1-(4-Benzyloxy-2-hydroxy-3-methylphenyl)ethylideneamino]guanidine, 1-[1-(Benzo[1,3]dioxol-5-yl)ethylideneamino]guanidine, 1-(3,4-Dichlorobenzylideneamino)guanidine, 1-[1-(4-Dimethylaminophenyl)pentylideneamino]guanidine, 1-{4-[Ethyl-(2-hydroxyethyl)amino]-2-methylbenzylideneamino}guanidine, 1-(4-Diethylamino-2-hydroxybenzylideneamino)guanidine, 1-(4-Diethylaminobenzylideneamino)guanidine, 1-[1-(4-Piperidin-1-yl-phenyl)ethylideneamino]guanidine, 1-{4-[Methyl-(2-cyanoethyl)amino]benzylideneamino}guanidine, 1-{4-[Methyl-(2-hydroxyethyl)amino]benzylideneamino}guanidine, 1-(4-Di-n-butylaminobenzylideneamino)guanidine, 1-(2-Methoxy-4-N,N-diethylaminobenzylideneamino)guanidine, 1-(3-Cyanobenzylideneamino)guanidine, 1-[(4-Trifluoromethyl)benzylideneamino]guanidine, 1-(2,4-Dimethoxybenzylideneamino)guanidine, 1-(2,3-Dimethoxybenzylideneamino)guanidine, 1-(4-Ethoxybenzylideneamino)guanidine, 1-(4-n-Propoxybenzylideneamino)guanidine, 1-(2,3,6-Trichlorobenzylideneamino)guanidine, 1-(4-Chlorobenzylideneamino)guanidine, 1-(5-Bromo-2-fluorobenzylideneamino)guanidine, 1-(2-Bromo-5-fluorobenzylideneamino)guanidine, 1-(3-Chlorobenzylideneamino)guanidine, 1-(3-Fluorobenzylideneamino)guanidine, 1-(2,3,4-Trimethoxybenzalideneamino)guanidine, 1-(3,5-Bistrifluoromethylbenzylideneamino)guanidine, 1-(5-Bromo-2,4-dimethoxybenzylideneamino)guanidine, and 1-[(5-(2-(Trifluoromethyl)phenyl)-furan-2-yl)-methyleneamino]guanidine (U.S. Patent Application No. 2005/0136444, hereby incorporated by reference, especially for the compounds disclosed therein). The compound may be selected from 1-(3,4-Dichlorobenzylideneamino)guanidine, 1-(3,4-Dibromobenzylideneamino)guanidine, and 1-(3-Bromo-4,5-dimethoxybenzylideneamino)guanidine (U.S. Patent Application No. 2005/0136444, hereby incorporated by reference, especially for the compounds disclosed therein).

The agent may be selected from N-(4-methyl-6-propyl-5,6,7,8-tetrahydro-quinazolin-2-yl)-guanidine, N-(6-isopropyl-4-methyl-5,6,7,8-tetrahydro-quinazolin-2-yl)-guanidine, N-(4-methyl-5,6,7,8-tetrahydro-quinazolin-2-yl)-guanidine, N-(4,5-dimethyl-5,6,7,8-tetrahydro-quinazolin-2-yl)-guanidine, N-(6-tert-butyl-4-methyl-5,6,7,8-tetrahydro-quinazolin-2-yl)-guanidine, N-(4-methyl-8-phenyl-5,6,7,8-tetrahydro-quinazolin-2-yl)-guanidine, N-(4-methyl-6-phenyl-5,6,7,8-tetrahydro-quinazolin-2-yl)-guanidine, N-[6-(1,1-dimethyl-propyl)-4-methyl-5,6,7,8-tetrahydro-quinazolin-2-yl]-guanidine, N-(8-tert-butyl-4-methyl-5,6,7,8-tetrahydro-quinazolin-2-yl)-guanidine, N-(4,6-dimethyl-5,6,7,8-tetrahydro-quinazolin-2-yl)-guanidine, N-(4-methyl-6,7,8,9-tetrahydro-5H-cycloheptapyrimidin-2-yl)-guanidine, N-(4-methyl-5,6,7,8,9,10-hexahydro-cyclooctapyrimidin-2-yl)-guanidine, N-(8-sec-butyl-4-methyl-5,6,7,8-tetrahydro-quinazolin-2-yl)-guanidine, N-(4,8-dimethyl-5,6,7,8-tetrahydro-quinazolin-2-yl)-guanidine, N-(8-allyl-4-methyl-5,6,7,8-tetrahydro-quinazolin-2-yl)-guanidine, N-(4-methyl-6,7-dihydro-5H-cyclopentapyrimidin-2-yl)-guanidine, N-(8-cyclohex-1-enyl-4-methyl-5,6,7,8-tetrahydro-quinazolin-2-yl)-guanidine, N-(6-isopropyl-5,6,7,8-tetrahydro-quinazolin-2-yl)-guanidine, N-(6-tert-butyl-5,6,7,8-tetrahydro-quinazolin-2-yl)-guanidine, N-(6-propyl-5,6,7,8-tetrahydro-quinazolin-2-yl)-guanidine, N-(6-phenyl-5,6,7,8-tetrahydro-quinazolin-2-yl)-guanidine, or N-(6-tert-butyl-4-trifluoromethyl-5,6,7,8-tetrahydro-quinazolin-2-yl)-guanidine, N-[8-(2-cyano-ethyl)-4-methyl-5,6,7,8-tetrahydro-quinazolin-2-yl]-guanidine, 2-guanidino-4-methyl-7,8-dihydro-5H-pyrido[4,3-d]pyrimidine-6-carboxylic acid tert-butyl ester, N-(6-phenyl-4-trifluoromethyl-5,6,7,8-tetrahydro-quinazolin-2-yl)-guanidine, N-(6-isopropyl-4-trifluoromethyl-5,6,7,8-tetrahydro-quinazolin-2-yl)-guanidine, N-(5,6,7,8-tetrahydro-quinolin-2-yl)-guanidine, N-(6-phenyl-5,6,7,8-tetrahydro-quinoline-2-yl)-guanidine, N-(5,6,7,8-tetrahydro-quinoxalin-2-yl)-guanidine, N-(6-phenyl-5,6,7,8-tetrahydro-quinoxalin-2-yl)-guanidine, N-(7-phenyl-5,6,7,8-tetrahydro-quinoxalin-2-yl)-guanidine, 6,7,8-tetrahydro-benzo[1,2,4]triazin-3-yl)-guanidine, N-(7-phenyl-5,6,7,8-tetrahydro-benzo[1,2,4]triazin-3-yl)-guanidine, and N-(6-phenyl-5,6,7,8-tetrahydro-benzo[1,2,4]triazin-3-yl)-guanidine (U.S. Pat. No. 7,544,691, hereby incorporated by reference, especially for the compounds disclosed therein). For example, the agent may be N-(6-isopropyl-4-methyl-5,6,7,8-tetrahydro-quinazolin-2-yl)-guanidine, N-(4-methyl-6-propyl-5,6,7,8-tetrahydro-quinazolin-2-yl)-guanidine, or N-(6-tert-butyl-4-methyl-5,6,7,8-tetrahydro-quinazolin-2-yl)-guanidine (U.S. Pat. No. 7,544,691, hereby incorporated by reference, especially for the compounds disclosed therein).

The compound may be selected from N-(4-methyl-6-propyl-5,6,7,8-tetrahydro-quinazolin-2-yl) guanidine, N-(6-isopropyl-4-methyl-5,6,7,8-tetrahydro-quinazolin-2-yl) guanidine, N-(4-methyl-5,6,7,8-tetrahydro-quinazolin-2-yl)-guanidine, N-(4,5-dimethyl-5,6,7,8-tetrahydro-quinazolin-2-yl)-guanidine and N-(6-tert-butyl-4-methyl-5,6,7,8-tetrahydro-quinazolin-2-yl)-guanidine, N-(4-methyl-8-phenyl-5,6,7,8-tetrahydro-quinazolin-2-yl) guanidine, N-(4-methyl-6-phenyl-5,6,7,8-tetrahydro-quinazolin-2-yl) guanidine, N-[6-(1,1-dimethyl-propyl)-4-methyl-5,6,7,8-tetrahydro-quinazolin-2-yl]guanidine, N-(8-tert-butyl-4-methyl-5,6,7,8-tetrahydro-quinazolin-2-yl) guanidine, N-(4,6-dimethyl-5,6,7,8-tetrahydro-quinazolin-2-yl)-guanidine, N-(4-methyl-6,7,8,9-tetrahydro-5H-cycloheptapyrimidin-2-yl) guanidine, N-(4-methyl-5,6,7,8,9,10-hexahydro-cyclooctapyrimidin-2-yl)-guanidine, N-(8-sec-Butyl-4-methyl-5,6,7,8-tetrahydro-quinazolin-2-yl) guanidine, N-(4,8-dimethyl-5,6,7,8-tetrahydro-quinazolin-2-yl) guanidine, N-(8-Allyl-4-methyl-5,6,7,8-tetrahydro-quinazolin-2-yl) guanidine, N-(6-Isopropyl-5,6,7,8-tetrahydro-quinazolin-2-yl)-guanidine, N-(6-tert-butyl-5,6,7,8-tetrahydro-quinazolin-2-yl)-guanidine, N-(4-methyl-6,7-dihydro-5H-cyclopentapyrimidin-2-yl)-guanidine, N-(6-propyl-5,6,7,8-tetrahydro-quinazolin-2-yl)-guanidine, N-(8-Cyclohex-1-enyl-4-methyl-5,6,7,8-tetrahydro-quinazolin-2-yl)-guanidine, N-(6-tert-Butyl-4-trifluoromethyl-5,6,7,8-tetrahydro-quinazolin-2-yl) guanidine, N-(6-phenyl-4-trifluoromethyl-5,6,7,8-tetrahydro-quinazolin-2-yl) guanidine, N-[8-(2-cyano-ethyl)-4-methyl-5,6,7,8-tetrahydro-quinazolin-2-yl]-guanidine, N-(6-Isopropyl-4-trifluoromethyl-5,6,7,8-tetrahydro-quinazolin-2-yl)-guanidine; 2-guanidino-4-methyl-7,8-dihydro-5H-pyrido[4,3-d]pyrimidine-6-carboxylic acid tert-butyl ester, N-(5,6,7,8-tetrahydro-quinolin-2-yl) guanidine, N-(6-phenyl-5,6,7,8-tetrahydro-quinolin-2-yl) guanidine, N-(5,6,7,8-tetrahydro-quinoxalin-2-yl) guanidine, N-(6-phenyl-5,6,7,8-tetrahydro-quinoxalin-2-yl)-guanidine, N-(7-phenyl-5,6,7,8-tetrahydro-quinoxalin-2-yl)-guanidine-5,6,7,8-tetrahydro-benzo[1,2,4]triazin-3-yl)-guanidine; N-(7-phenyl-5,6,7,8-tetrahydro-benzo[1,2,4]triazin-3-yl)-guanidine, and N-(6-phenyl-5,6,7,8-tetrahydro-benzo[1,2,4]triazin-3-yl)-guanidine (U.S. Pat. No. 7,544,691, hereby incorporated by reference, especially for the compounds disclosed therein).

The agent may be selected from any one of Formulas I-X, or a derivative, ester, or salt thereof:

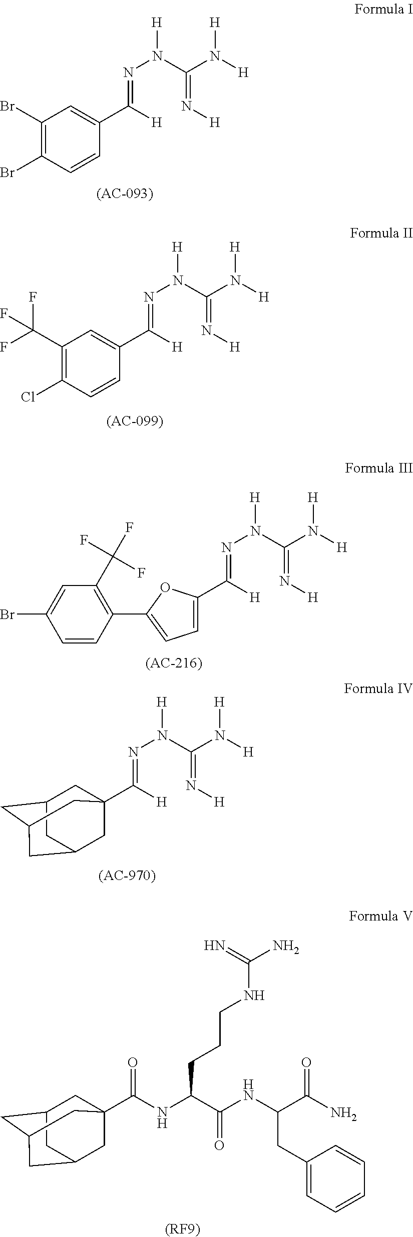 [SMILES: [H]/N=C(\N)N/N=C/c1ccc(Br)c(Br)c1 ; [H]/N=C(\N)N/N=C/c1ccc(Cl)c(C(F)(F)F)c1 ; [H]/N=C(\N)N/N=C/c1ccc(-c2ccc(Br)cc2C(F)(F)F)o1 ; [H]/N=C(\N)N/N=C/C12CC3CC(CC(C3)C1)C2 ; N=C(N)NCCC[C@H](NC(=O)C12CC3CC(CC(C3)C1)C2)C(=O)NC(Cc1ccccc1)C(N)=O ; "(RF9)"]

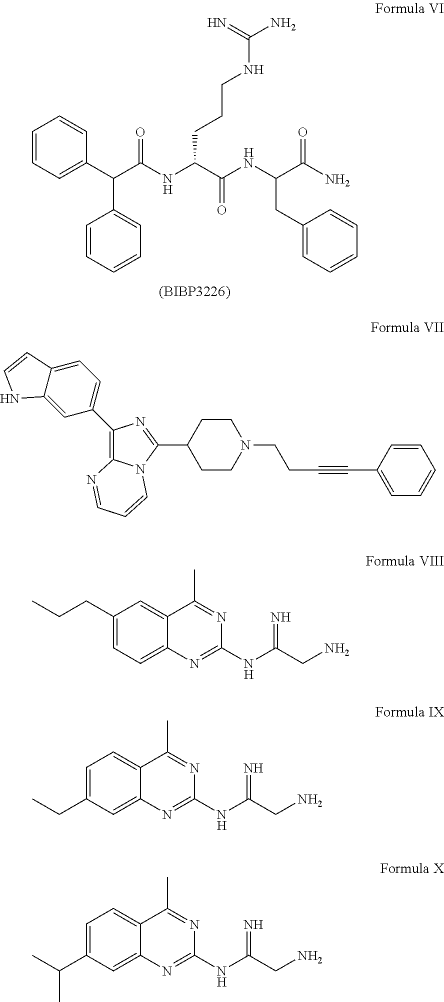 [SMILES: N=C(N)NCCC[C@@H](NC(=O)C(c1ccccc1)c1ccccc1)C(=O)NC(Cc1ccccc1)C(N)=O ; C(#Cc1ccccc1)CCN1CCC(c2nc(-c3ccc4cc[nH]c4c3)c3ncccn23)CC1 ; CCCc1ccc2nc(NC(=N)CN)nc(C)c2c1 ; CCc1ccc2c(C)nc(NC(=N)CN)nc2c1 ; Cc1nc(NC(=N)CN)nc2cc(C(C)C)ccc12]

For example, the agent may be a molecule disclosed in PCT Patent Application Publication No. WO 2009/038012 (hereby incorporated by reference, especially for the compounds disclosed therein), including molecules similar to the molecule depicted in Formula VII. Similarly, the agent may be a molecule disclosed in U.S. Patent Application Publication No. U.S. 2003/0139431 (hereby incorporated by reference, especially for the compounds disclosed therein), including molecules similar to the molecules depicted in Formulas VIII-X.

Certain embodiments of the present invention are well adapted to carry out the objects and obtain the ends and advantages mentioned, as well as those inherent therein. The embodiments described herein are not intended as limitations on the scope of the invention.

### EXEMPLIFICATION

The present description is further illustrated by the following examples, which should not be construed as limiting in any way. The contents of all cited references (including literature references, issued patents, and published patent applications as cited throughout this application) are hereby expressly incorporated by reference. When definitions of terms in documents that are incorporated by reference herein conflict with those used herein, the definitions used herein govern.

### Example 1

### C. elegans Strains Used in Examples 2 to 8

### Nematode Growth.

Strains were grown, unless indicated otherwise, on nematode growth medium (NGM) 2% agar plate seeded with E. coli strain OP50 and maintained at 20° C. under standard animal husbandry conditions (Brenner, Genetics 77, 71 (1974)).

### Strains.

The wild-type worm strain was the Bristol variety, N2. Mutation strains were obtained from the Caenorhabditis Genetics Center (CGC) including RB761 npr-7(ok527) X and RB1405 npr-22(ok1598) IV, which were provided by the C. elegans Gene Knockout Project at OMRF (http://www.mutantfactory.ouhsc.edu/), and VC1971 flp-14(gk3109) III, which was provided by the C. elegans Reverse Genetics Core Facility at the University of British Columbia (http://www.celeganskoconsortium.omrf.org). Mutation strain FX02427flp-13(tm2427) IV was obtained from the National Bioresource Project.

Mutant Strains:
FX02427 FLP-13(2427) IV
VC1971 FLP-24(GK3109) III
RB761 NPR-7(OK527) X
RB1405 NPR-22(OK1598) IV
RB1990 FLP-7(OK2625) X
TB528 CEH-14(CH3) X

### Mutant Alleles:

flp-13(tm2427) is a 382 by deletion (89:291) starting in the first intron to part of the second intron that removes the second exon and causes a frame shift at amino acid residue 31. This mutation deletes the C-terminus of the propeptide and completely abolishes all peptide cleavage sites and synthesis of all FLP-13 peptides.

flp-24(gk3109) is a 1180 by deletion (92:210) starting in the first intron to the 3′ intergenic sequence that removes the second exon and 3′UTR of flp-24. This deletion truncates the protein at amino acid residue 29, removing the majority of the propeptide, all post-translational processing sites, and the FLP-24 peptide.

npr-22(ok1598) is a 2528 by deletion that erases exons 2 to 5 of isoform a and exons 2 to 6 of isoform b, causing protein truncation before the first transmembrane domain and resulting in a null mutation of both isoforms.

npr-7(ok527) Xis a 1211 by deletion that erases half of exon 3 and the entire exon 4.

### Example 2

### Generation of Transgenic Lines

### Heat-Shock Transgenic Lines.

Conditional expression of cDNAs was achieved by generating a fusion of the coding sequence of a gene under study to the hsp-16.41 promoter (Lesa and Sternberg, Mol. Biol. Cell 8, 779-793 (1997)). A synthetic DNA fragment consisting of the hsp-16.41 promoter, DNA coding sequence and its endogenous 3′UTR was generated using fusion PCR techniques (Hobert, BioTechniques 32, 728 (2002)). N2 animals of mixed stages were harvested for RNA extraction and subsequently reverse-transcribed into cDNA for amplification of flp-13 cDNA and flp-24 cDNA. The 3′UTR regions were amplified from N2 wild-type mixed stage animal genomic lysates. The hsp-16.41 promoter region was amplified from plasmid ppD49.83 and the PCR product was verified by DNA sequencing. The cDNAs and 3′UTR genomic DNAs amplified were sequenced and blasted against the predictions from www.wormbase.org. The final fusion PCR product was sequenced and blasted against the predicted DNA sequence for the conditional gene overexpression assay.

hs:FLP-24 overexpression DNA was used as a backbone to introduce exogenous zebrafish RF-amides in C. elegans. The FLP-24 peptide was replaced by either zebrafish NPFF (DrNPFF) or zebrafish RFRP-1 (DrRFRP-1), keeping the endogenous FLP-24 signal peptide, propeptide, cleavage sites, and 3′UTR intact.

Primer sequences used in the generation of the above fusions were as follows:

P200 Phsp-16.41 forward:
SEQ ID NO: 1
ATGACCATGATTACGCCAAG
P201 Phsp-16.41 reverse:
SEQ ID NO: 2
GCTAGCCAAGGGTCCTCCT
P207 hlh-13 forward:
SEQ ID NO: 3
AGGAGGACCCTTGGCTAGCATGACAGCTTCATCTTCTGGGTGT
P206 hlh-13 reverse:
SEQ ID NO: 4
TAATCAGTATGTTTATTGAAATGAAAGATAGAAAATCATGAGTTGTAT
TCGTG
P249 flp-7 forward:
SEQ ID NO: 5
AGGAGGACCCTTGGCTAGCATGCTTGGATCCCGCTTC
P250 flp-7 reverse:
SEQ ID NO: 6
AACAGGCGTCGGTTCTTTATTT
P245 flp-13 forward:
SEQ ID NO: 7
AGGAGGACCCTTGGCTAGCATGATGACGTCACTGCTCACT
P247 flp-13 reverse:
SEQ ID NO: 8
TTATTTTCTGCCAAAACGAATG
P209 flp-24 forward:
SEQ ID NO: 9
AGGAGGACCCTTGGCTAGCATGTTGTCGTCGCGCACATCGTCCATCAT
P212 flp-24 reverse:
SEQ ID NO: 10
TCAGATGCTTCTTTTTCCAAATC
P283 Danio rerio NPFF forward:
SEQ ID NO: 11
GTGCTGCACCAGCCTCAGCGGTTTGGAAAAAGAAGCATCTGATAATAT
ACCATCTACC
P282 Danio rerio NPFF reverse:
SEQ ID NO: 12
GATGCTTCTTTTTCCAAACCGCTGAGGCTGGTGCAGCACACGTTTGTG
TGGAATCTCTCC
P280 Danio rerio RFRP-1 forward:
SEQ ID NO: 13
CCAGCTCACCTGCATGCAAACCTCCCTCTTCGCTTTGGAAAAAGAAGC
ATCTGATAA
P279 Danio rerio RFRP-1 reverse:
SEQ ID NO: 14
AGCGAAGAGGGAGGTTTGCATGCAGGTGAGCTGGACGTTTGTGTGGAA
TCTCTCC-3′

To synthesize the fusion PCR fragments for heat shock overexpression, the following primer pairs were used:
hs:FLP-13: P200 and P247
hs:FLP-24: P200 and P212
hsFLP-7: P200 and P250
hs:HLH-13: P200 AND P206
hs:DrNPFF: P200 and P212
hs:DrRFRP-1: P200 and P212

### Example 3

### Expression Patterns in Transgenic Strains

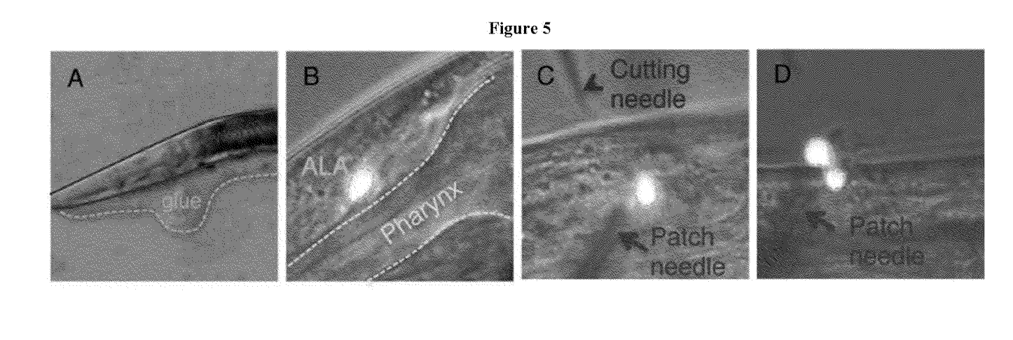

To identify neuropeptides that mediate ALA-dependent sleep, micro-dissected GFP-labeled ALA neurons during late L4-stage were profiled (FIG. 5). Expression patterns of a gene of interest were examined by generating a fusion of the conserved non-coding sequence in the vicinity of a coding sequence to a basal promoter, pes-10, attached with a fluorescent protein coding sequence and unc-54 3′UTR (Hobert, BioTechniques 32, 728 (2002); Kuntz et. al., Genome Res. 18(12), 1955 (2008)). The genomic sequences of C. elegans, C. briggsae, C. remanei, and C. brenneri were obtained from WormBase (www.wormbase.org) and compared for conservation at or higher than 67% sequence identity.

All conserved sequences were amplified from N2 wild-type mixed stage worm genomic lysates. The pes-10, green fluorescent protein (GFP), and unc-54 3′UTR sequence was amplified from pD97.78. The mCherry sequence was amplified from pAM-31.1.

Primer sequences used in the generation of the above fusions are listed as follows:

P65 Ppes-10::GFP forward:
SEQ ID NO: 15
CTAGCAAAAATGCATAAGG
P1 Ppes-10::GFP reverse:
SEQ ID NO: 16
GTGTCAGAGGTTTTCACCGTCA
P85 mCherry forward:
SEQ ID NO: 17
CTGTAATTTTTAACTTTCAGATGGTCTCAAAGGGTGAAGAA
P86 mCherry reverse:
SEQ ID NO: 18
CTACTTATACAATTCATCCATGCCACCT
P157 Ppes-10 promoter forward:
SEQ ID NO: 19
CTAGCAAAAATGCATAAGG
P158 Ppes-10 promoter reverse:
SEQ ID NO: 20
TTTTTCTACCGGTACCTTACGCTTC
P253 PflP-13 forward:
SEQ ID NO: 21
CATCGTCGTAAAAACAAATTCAA
P254 pflP-13 reverse:
SEQ ID NO: 22
CCTTATGCATTTTTGCTAGTTTGACACAAAATGCCGACT
P242 Pflp-24 forward:
SEQ ID NO: 23
CATCCAATATGGTGAGTTTCTCTG
P243 pflP-24 reverse:
SEQ ID NO: 24
CCTTATGCATTTTTGCTAGCGTCTGAAATTTCGAAAAGTAATAAT
P260 Pnpr-22 forward:
SEQ ID NO: 25
AGTTTTGAAGCTTACTTGACATGAA
P261 Pnpr-22 reverse:
SEQ ID NO: 26
CCTTATGCATTTTTGCTAGAGGGAAGGGAAAAACTTGAA
P89 Pver-3::mCherry forward:
SEQ ID NO: 27
TGTTTTCAAAGTGTTGGAATCAAT
P90 Pver-3::mCherry reverse:
SEQ ID NO: 28
AAAAATCGATCCTGCAGGCGAACCGAACCGAATGAAACA

### Extrachromosomal Transgenes.

Transgenic strains were generated by injecting the fusion DNA fragments into the gonads of young adult hermaphrodites along with a fluorescent co-injection marker and a carrier (Mello and Fire, Methods Cell Biol. 48, 451 (1995)). In order to control for variation between transgenes, at least two independent lines from each injection were used for behavioral assays and expression patterns.

### Transgenic Strains:

TB513: dpy-20(e2017); chIs513[ceh-14::GFP, dpy-20(+)]
PS5009: pha-1(e2123ts); him-5(e1490); syEx723[hsp16-41: lin-3C cDNA(10 ng/μl)+myo2:GFP(10 ng/μl)+pha-1(+)(pbx-1)(90 ng/μl)+bluescript(90 ng/μl)]
PS6562 syEX1285[hsp16-41:flp-13(10 ng/μl), PmyosdsRed(10 ng/μl), KS+(90 ng/μl)]
PS6563 syEX1286[hsp16-41:flp-24(10 ng/μl), PmyosdsRed(10 ng/μl), KS+(90 ng/μl)]
PS6564 syEX1287[hsp16-41:flp-13; flp-24(10 ng/μl), PmyosdsRed(10 ng/μl), KS+(90 ng/μl)]
PS6565 RB1405 npr-22(ok1598)+syEX1288[hsp16-41:flp-13; flp-24(10 ng/μl), PmyosdsRed(10 ng/μl), KS+(90 ng/μl)]
PS6566, PS5009 syIs197, him-5(e1490); FX02427 flp-13(tm2427)
PS6567, PS5009 syIs197, him-5(e1490); VC9171 flp-24(gk3109)
PS6568, PS5009 syIs197, him-5(e1490); RB1405 npr-22(ok1598)
PS6569 syEx1285, syIs197, him-5(1490); ceh-14(ch3) X
PS6570 syEx1286, syIs197, him-5(1490); ceh-14(ch3) X
PS6571 syEX1294[hsp16-41:flp-7(10 ng/μl), PmyosdsRed(10 ng/μl), KS+(90 ng/μl)]
PS6572 syEX1295[Pflp-13::GFP(10 ng/μl), PmyosdsRed(10 ng/μl), KS+(90 ng/μl)]
PS6573 syEX1296[Pflp-24::GFP(10 ng/μl), PmyosdsRed(10 ng/μl), KS+(90 ng/μl)]
PS6574 syEX1297[Pnpr-22::GFP(10 ng/μl), PmyosdsRed(10 ng/μl), KS+(90 ng/μl)]
PS6575 syEX1298[Prab-3:npr-22::GFP(10 ng/μl), PmyosdsRed(10 ng/μl), KS+(90 ng/μl)]
PS6576 syEX1299[Pmyo-3:npr-22::GFP(10 ng/μl), PmyosdsRed(10 ng/μl), KS+(90 ng/μl)]
PS6577 syEX1300[hsp16-41:DrRFRP-1(10 ng/μl), PmyosdsRed(10 ng/μl), KS+(90 ng/μl)]
PS6578 syEX1301[hsp16-41:DrNPFF-1(10 ng/μl), PmyosdsRed(10 ng/μl), KS+(90 ng/μl)]

### Single ALA Neuron Dissection and Transcriptome Profiling.

Individual ALA neurons from wild-type larvae of strain TB513 at the mid-L4 larval stage were hand-picked and glued on an agar pad for micro-dissection (Schwarz et. al., Proc. Natl. Acad. Sci. U.S.A. 109(40), 16246 (2012)). Isolated individual ALA neurons tagged by GFP were collected with an unpolished patch-clamp tube that served as a pipette, transferred to a prelubricated eppendorf tube, and snap-frozen with liquid nitrogen. Frozen tubes with individual ALA neurons were kept at −70° C. until amplification.

RT-PCR of individual neurons was performed essentially as in Schwarz et al. (Proc. Natl. Acad. Sci. U.S.A. 109(40), 16246 (2012)). To obtain RNA-seq data by Illumina sequencing, aliquots of individual RT-PCR reactions were pooled into two pools (4 cells and 5 cells). Existing whole wild-type larval RNAseq data (Schwarz et al., Proc. Natl. Acad. Sci. U.S.A. 109(40), 16246 (2012)) was used as a control for housekeeping versus ALA-enriched genes. Expression values for genes were defined by pooling reads from both wild-type mid-L4 ALA neuron sets into a single set of expression values, doing likewise for both wild-type larval RNA-seq sets, and computing ALA/larval ratios of gene activity. After quality filtering and truncation but before mapping, RNA-seq data from the two pools of wild-type ALA comprised 1,164,892,280 nt in 30,655,060 reads and 1,520,526,262 nt in 40,013,849 reads (all reads were single-end and 38 nt). Of these, 25.2% could be mapped to WS190 protein-coding gene models (i.e., 17,798,207 out of 70,668,909 reads). The expression of 7,698 and 4,068 genes were detected in the two pools separately, and 8,133 genes collectively were detected.

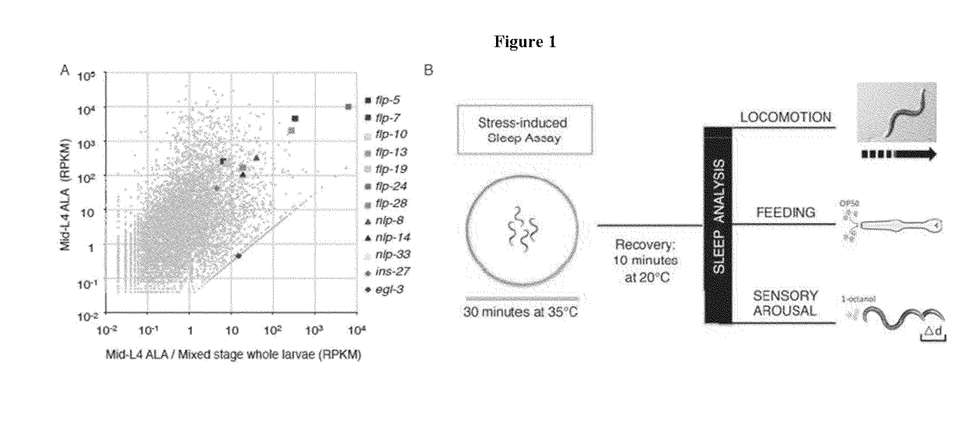

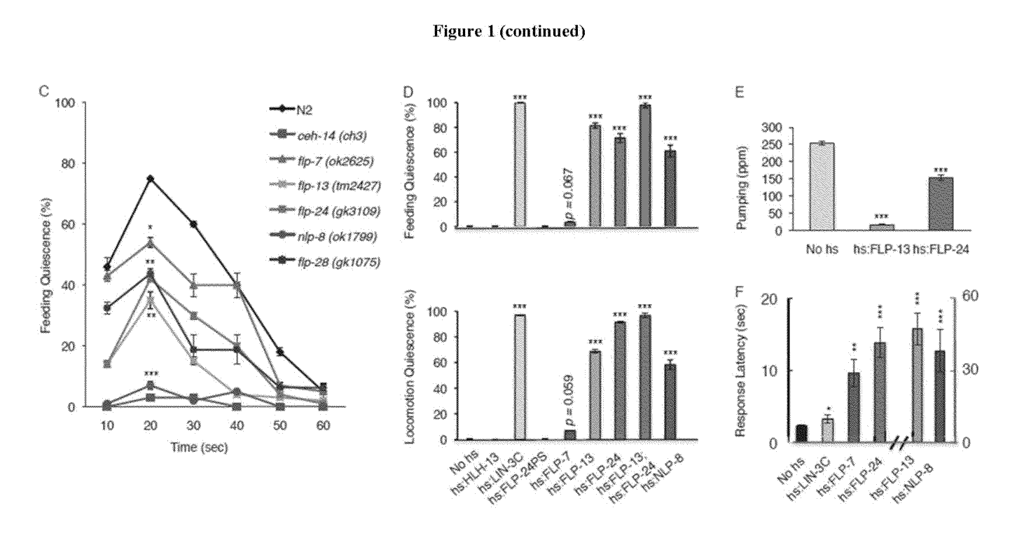

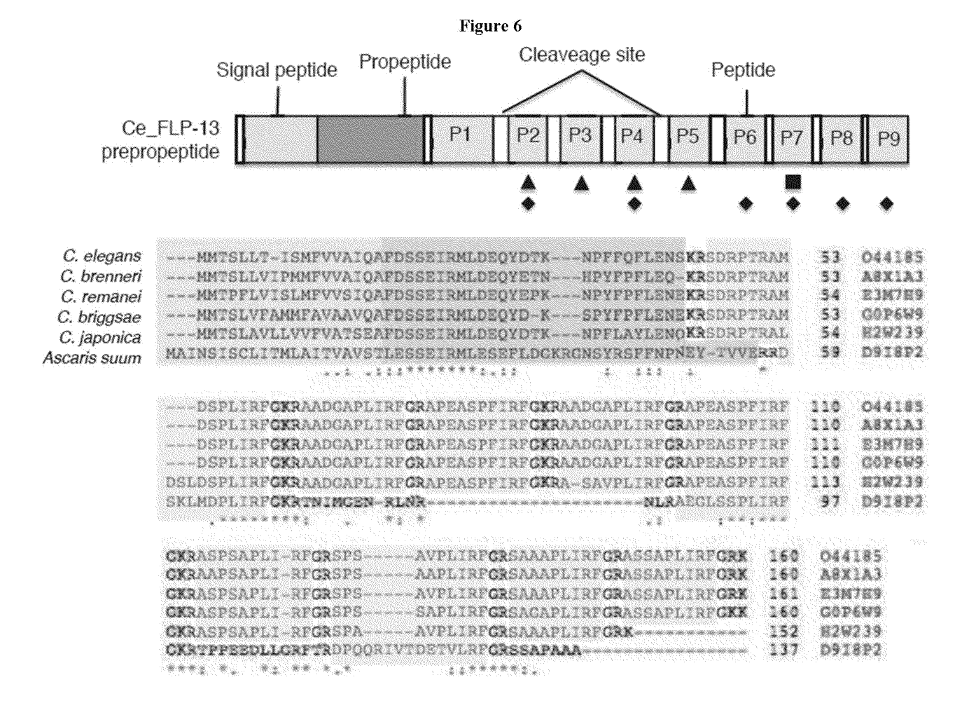

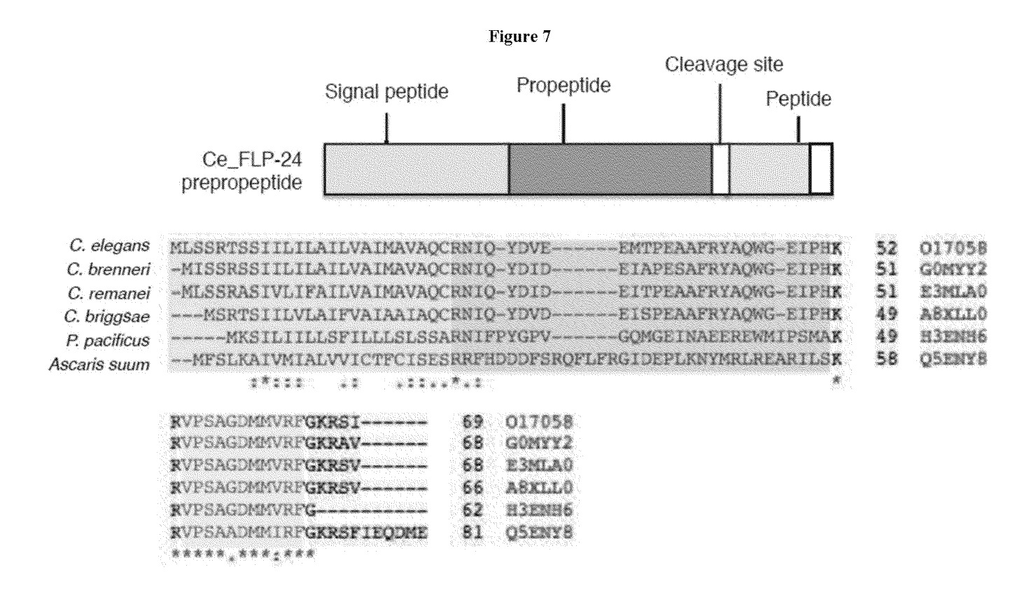

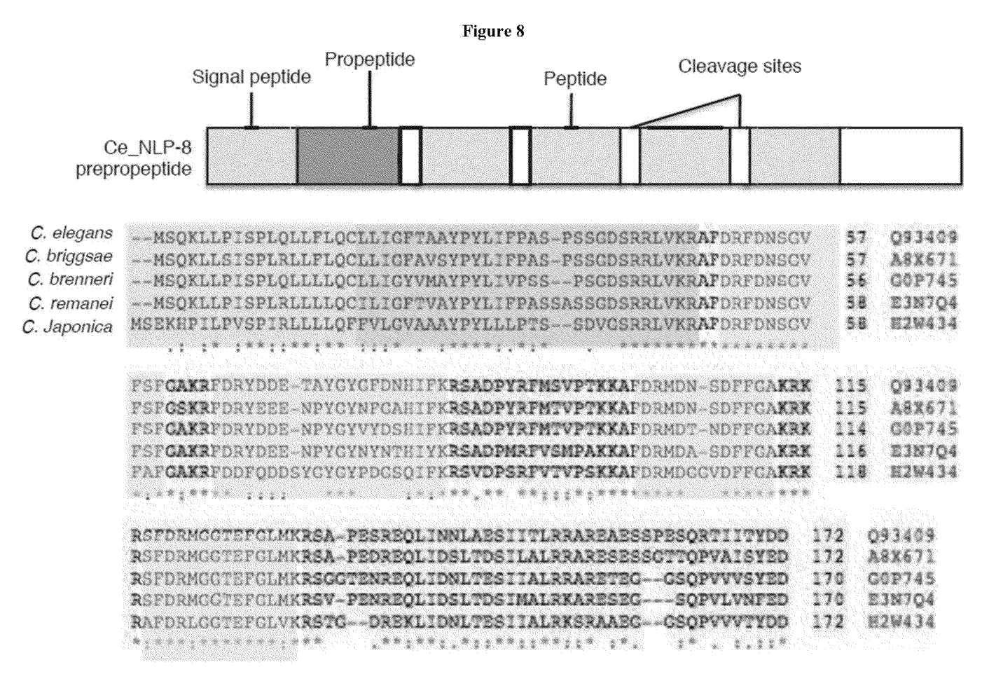

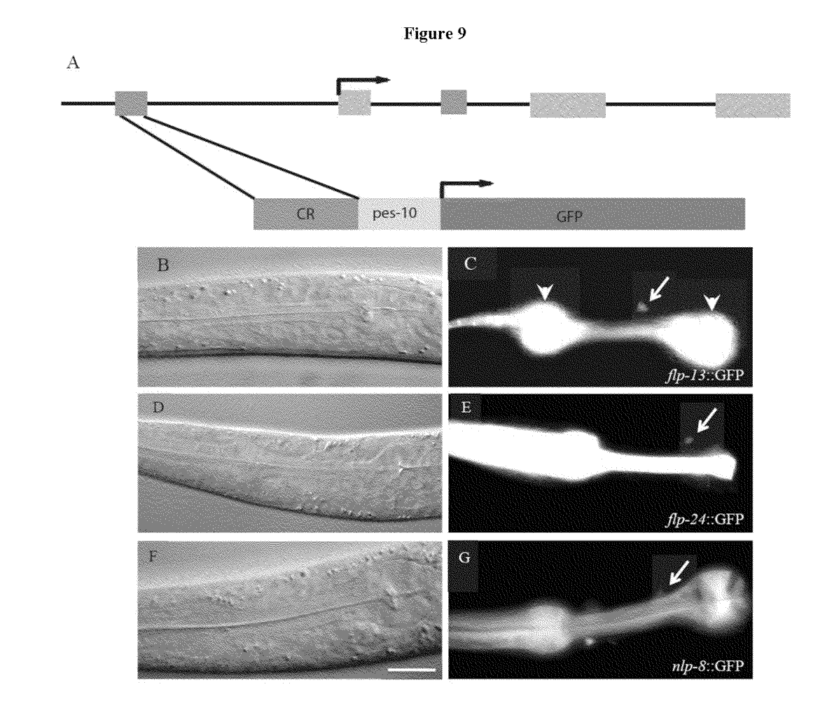

Deep sequencing analysis allowed the detection of 8,113 protein-coding genes, with eight genes encoding neuropeptides that had greater than 17-fold enrichment in ALA relative to whole larval transcriptome (FIG. 1, panel (A); Table 1). The majority of these genes belonged to the FMRF-amide like neuropeptide (FLP) family, which are the most diverse peptides in nematodes. Of the highest ALA-enriched genes, FMRFamide-like peptides flp-24, flp-13, and a previously known ALA-expressed gene flp-7, were found to be expressed. nlp-8, found in the neuropeptide-like-protein (NLP) family, was also highly enriched in ALA. These genes encode prepropetides of multiple mature neuropeptides (FIGS. 6, 7, and 8). The expression of flp-13, flp-24 and nlp-8 in ALA was verified during all larval stages and the adult stage by green fluorescence protein (GFP) reporter constructs (FIG. 9).

TABLE 1
Eight neuropeptides have at least 17-fold higher expression in the ALA
relative to whole larvae.

| | | | ALA v.s. |
|---|---|---|---|
| Genes | ALA | Whole larvae | Whole larvae |
| flp-24 | 10176.34 | 1.59 | 6400.21 |
| flp-7 | 4518.55 | 13.51 | 384.46 |
| flp-13 | 2063.11 | 7.52 | 274.35 |
| nlp-8 | 332.58 | 8.25 | 40.31 |
| nlp-33 | 109.35 | 4.58 | 23.88 |
| flp-28 | 169.94 | 8.76 | 19.4 |
| nlp-14 | 106.87 | 5.56 | 19.22 |
| flp-10 | 74.42 | 4.25 | 17.51 |

### Example 4

### EGF Heat-Induced Feeding Suppression Assays

To identify if these ALA-enriched neuropeptides mediate sleep, the neuropeptides were examined to determine whether they could confer resistance to EGF/stress-induced sleep. Ten young adult hermaphrodites were hand selected and transferred by a platinum pick to a NGM plate coated with a thin lawn of OP50 bacteria. The plates were sealed with parafilm and placed in a 35° C. water bath for 30 min, recovered at 20° C. for 10 min before examination for feeding for 60 min, as indicated by pharyngeal pumping activity. Each animal was examined under high magnification on a stereomicroscope for 5 sec for feeding, indicated by the movement of the grinder in the posterior pharyngeal bulb. Typically, animals with no observed movement were scored as quiescence for feeding behavior.

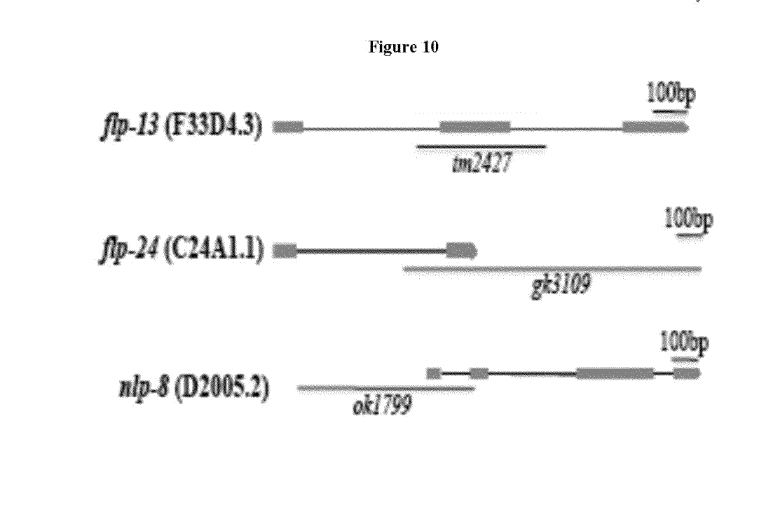

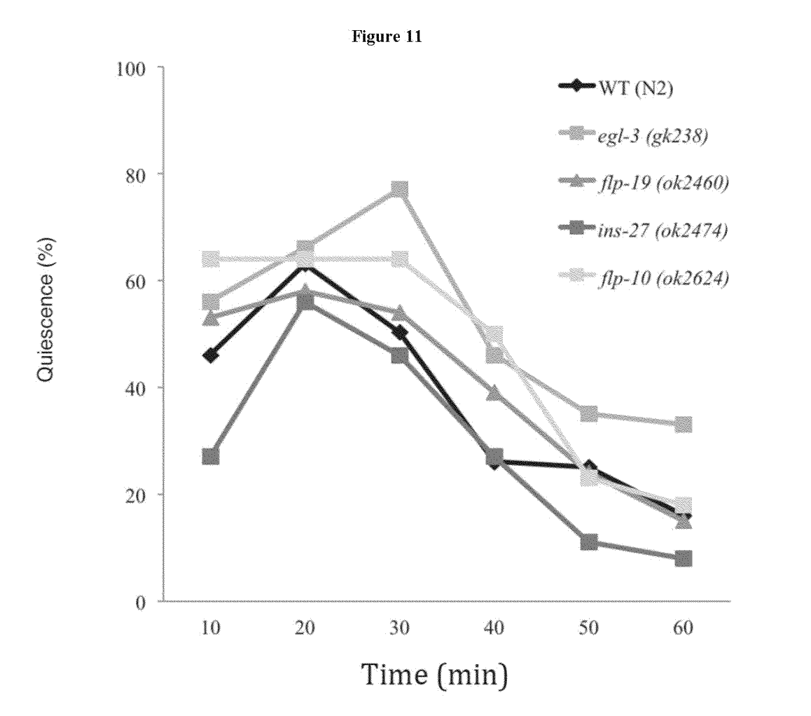

Mutants of all four highest scoring ALA-synthesized neuropeptides were resistant to EGF/stress-induced sleep to varying degrees: nlp-8 exhibited the highest resistance, followed by flp-13, flp-24 and flp-7 mutants. By contrast, resistance in other mutants of ALA-synthesized neuropeptides were not observed, namely, flp-10, flp-19 and ins-27 (FIGS. 10 & 11; Table 2). These results suggest that ALA induces sleep through the combined effects of a set of at least four neuropeptides.

TABLE 2
Mutant strains tested for resistance to EGF-induced sleep

| Strain | Number | EGF-resiatant |
|---|---|---|
| N2 | 200 | − |
| ceh-14 (ch3) | 50 | + |
| egl-3 (gk238) | 50 | − |
| flp-5 (gk3123) | 120 | + |
| flp-7 (ok2625) | 50 | + |
| flp-13 (tm2427) | 120 | + |
| flp-10 (ok2624) | 50 | − |
| flp-19 (ok2460) | 50 | − |
| flp-24 (gk3109) | 100 | + |
| flp-28 (gk1075) | 50 | + |
| ins-27 (ok2474) | 50 | − |
| nip-8 (ok1799) | 100 | + |

### Example 5

### Physiological Behavior Measurements

To characterize the role of ALA-synthesized neuropeptides in inducing C. elegans sleep, three physiological behavior measurements were assessed: locomotor cessation, feeding cessation, and decreased sensory arousal to stimulation (FIG. 1, panel (B)).

Neuropeptide Heat-Activated Gain-of-Function Quiescence Assays.

Young adult hermaphrodites were hand selected and transferred by a platinum pick to NGM plates coated with a thin lawn of OP50 bacteria. Each plate hosted approximately 20 animals. Heat shock assays were performed as described by Van Buskirk & Sternberg (Development 137(12), 2065 (2010)). Briefly, the plates were sealed with parafilm and placed in a 33° C. water bath for 30 min, returned to 20° C. for 2 h, and scored for suppression of locomotion and feeding, and for response latency to sensory and mechanical stimulations.

Feeding Behavior.

Each animal was examined for feeding, indicated by the movement of the grinder in the posterior pharyngeal bulb under high magnification on a stereomicroscope for 5 sec. Typically, animals with no movement observed were scored as quiescent for feeding behavior.

Locomotion.

For locomotion analysis, animals recovered from heat-activation were placed on a Leica stereomicroscope base with a Unibrain camera, illuminated with continuous white light, and imaged for 4 minutes to track forward and backward movements. The centroid velocity plot was calculated over a 2-s interval (Van Burskirk & Sternberg, Development 137(12), 2065 (2010)).

Of the four neuropeptides conditionally expressed under a heat-shock promoter, three were sufficient to induce sleep in typically active young adults (FIG. 1, panels (C), (D), & (E)). Additionally, animals with heat-activated neuropeptides had reduced body curvature. hs:FLP-13 animals displayed brief bouts of movement wherein their bodies jerked backward, while hs:FLP-24 animals rarely moved at all. Overexpression of both FLP peptides further augmented the effect and completely abolished or reduced feeding as indicated by pharyngeal pumping rate (FIG. 1, panels (D) & (E)).

In agreement with the mild resistance to stress-induced sleep in flp-7 mutants, mild induction of sleep by overexpressing FLP-7 peptides was also observed (FIG. 1, panel (D)). Overexpression of NLP-8, however, only induced sleep in approximately half of the animals

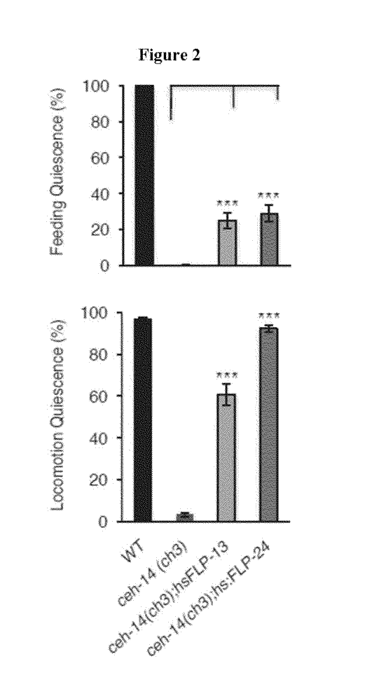

(FIG. 1, panel (D)). Thus, the neuropeptide differences in inducing sleep may be due to functional redundancy. Indeed, both hs:FLP-13 or hs:FLP-24 were capable of partially restoring sleep in ceh-14 null mutants (FIG. 2). The transcription factor, ceh-14, is essential for proper development of the ALA and is critical for the expression of LET-23/EGFR in the ALA, and ceh-14 null mutants are completely resistant to EGF/stress-induced sleep (FIG. 1C). These results suggest that the neuropeptides function synergistically downstream of EGF, and are sufficient to induce sleep when expressed alone or together.

Chemical Response Assay.

C. elegans move immediately backward when presented with 30% 1-octanol; however, such withdrawal behavior may be delayed due to decreased sensory arousal during sleep. For the chemical response assay, animals recovered from heat-activation were presented with 30% 1-octanol (Raizen et al., Nature 451(7178), 569 (2008)). 1-octanol was diluted to a final concentration of 30% with Ethanol (volume:volume). Briefly, an eyebrow hair dipped in 30% 1-octanol was presented to the nose of each worm within the length of a pharynx. The time required for the worm to move backward for the length of a pharynx was recorded and documented as response latency. All comparison between treatments and genotypes were made on the same day. Similar to sleeping animals, hs:FLP-13 and hs:FLP-24 animals exhibited delayed withdrawal behavior when presented with 1-octanol (FIG. 1, panel (F)).

Lethargus Analysis.

For lethargus analysis, mid-L4 animals were handpicked and tracked for movement over the next 7 h. The animals were inspected for vulval eversion at the end of recording to ensure that they had reached the young adult stage. Movement was quantified by calculating the centroid velocity (Van Burskirk & Sternberg, Development 137(12), 2065 (2010)).

Mechanical Response Assay.

For the mechanical response assay, animals were perturbed by harsh touch with the tip of a platinum worm pick on the nose or tail of the worm. Animals were recorded for forward and backward movements under a Leica stereomicroscope with a Unibrain camera.

### Example 6

### Elucidation of Signaling Pathways

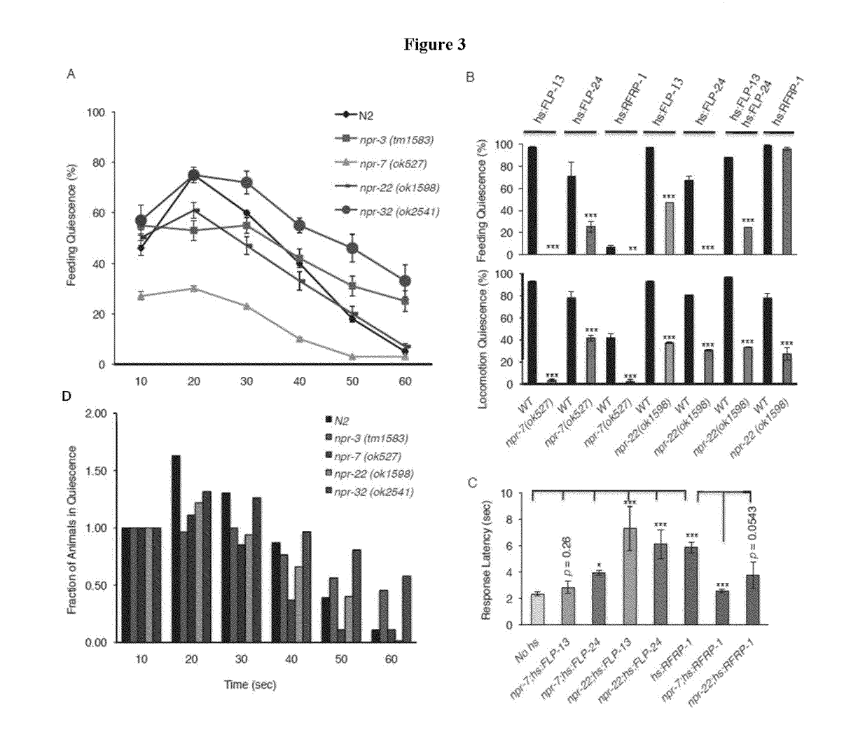

Neuropeptides exert their effects predominantly through G-protein-coupled receptors (GPCR). Four ALA-expressed FLP genes, flp-7, flp-9, flp-13, flp-22, can activate the GPCR NPR-22 in vitro (Table 3), suggesting that NPR-22 might act as a receptor for ALA neuropeptides. Thus, the responses of npr-22 and mutants of paralogous receptors to EGF/stress-induced sleep were investigated. Heat-stressed mutants defective in npr-3, npr-7, or npr-22 displayed reduced sleep relative to wild type animals (FIG. 3, panel (A)). Sleep and feeding are mutually exclusive behaviors, and because the effect of feeding suppression is prominent and robust, the feeding effect induced by conditionally expressed neuropeptides was also ascertained. All peptides tested required the presence of intact npr-7 and npr-22 (FIG. 3, panel A). Remarkably, the absence of the NPR-7 receptor completely restored feeding for hs:FLP-13 and partially restored feeding for hs:FLP-24. The mutation of npr-22 restored feeding by hs:FLP-13 peptides partially and by hs:FLP-24 peptides completely.

TABLE 3
C.elegans RFamide peptides that can activate
calcium response via the G-protein coupled
receptor NPR-22 (SEQ ID NOS 29-42,
respectively, in order of appearance)

| | | Activation |
|---|---|---|
| Gene | Peptide Sequence | of NPR-22 |
| flp-7 | SPMQRSSMVRF | + |
| | TPMQRSSMRF | + |
| | SPMERSAMVRF | + |
| | SPMDRSKMVRF | + |
| flp-9 | KPSFVRF | + |
| flp-13 | AMDSPLIRF | − |
| | AADGAPLIRF | + |
| | APEASPLIRF | − |
| | ASPSAPLIRF | + |
| | SPSAVPLIRF | + |
| | ASSAPLIRF | + |
| | SAAAPLIRF | + |
| flp-22 | SPSAKWMRF | + |
| flp-24 | VPSAGDMMVRF | N/A |

Next, the ambulatory effects were assayed and robust locomotor rescue was observed in npr-7 mutants with hs:FLP-13 peptides, and partial rescue was observed for hs:FLP-24, suggesting that NPR-7 is likely a receptor of FLP-13 peptides. Additionally, npr-22 was found to be partially required to mediate locomotion suppression by all peptides tested (FIG. 3, panel (B)). Consistent with resumed motor activity, animals lacking both GPCRs (NPR-7 and NPR-22) resumed sensory perception in the presence of extra FLP peptides (FIG. 3, panel (C)). These results demonstrate that ALA-synthesized neuropeptides act through multiple GPCRs to induce sleep. In fact, multiple aspects of behavior are affected during sleep, and to the extent analyzed, multiple levels in sensory processing are dampened, allowing multiple peptide-receptor combinations to induce at least partial sleep phenotypes.

### Example 7

### The Expression of Vertebrate Neuropeptides in C. elegans

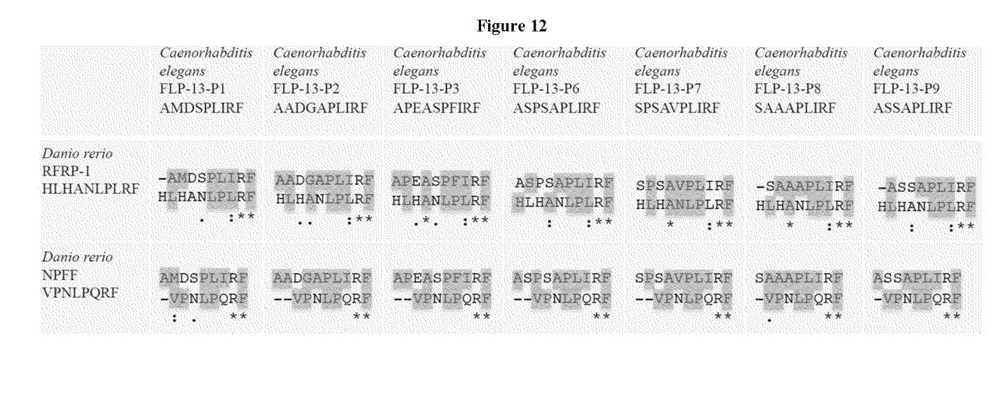

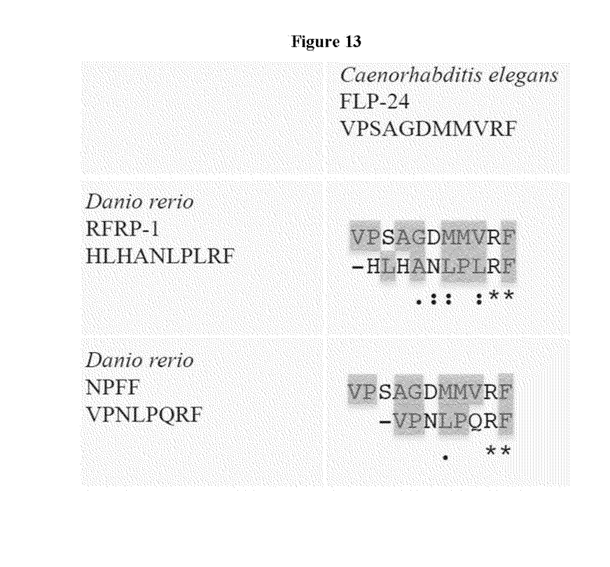

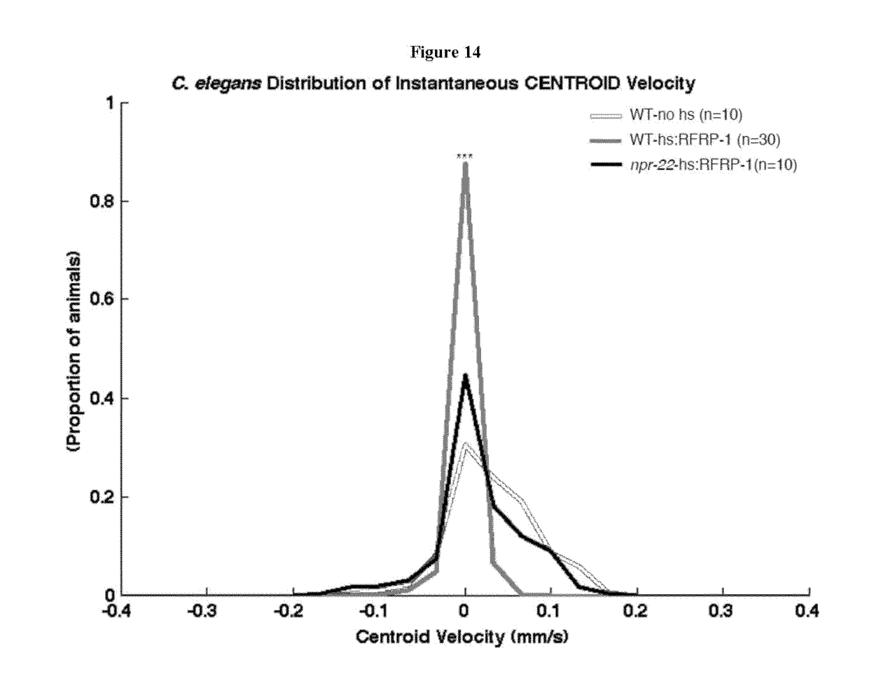

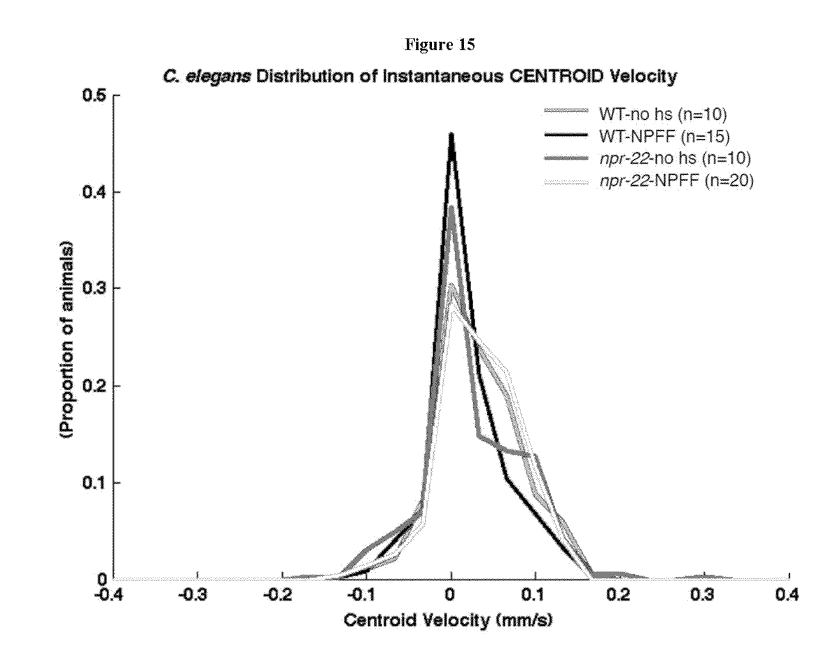

NPR-7 shares high amino acid sequence similarity with human FF1 receptor (NPFFR1) and NPY1R, while the two isoforms of NPR-22 are highly similar in sequence with human FF1 receptor NPFFR1, and NPY1R (Table 4). Reciprocal BLAST against vertebrate receptors revealed that NPR-7 and NPR-22 are likely orthologs of human FF1 and zebrafish npffr1. The bioactive peptide sequence of FF1 receptor ligands generated by the prepropeptide genes NPVF and NPFF, with FLP-13 and FLP-24 peptides were aligned, and a conserved carboxy terminus and similar hydrophobic backbone between FLP peptides and RFRP-1 was observed (FIGS. 12 & 13). To test the potential relevance of amino acid sequence similarity, FLP-24 peptide was substituted with zebrafish RFRP-1 or NPFF, and the FLP-24 backbone was used to drive conditional expression of these peptides in C. elegans. Zebrafish RFRP-1 produced a sleep-inducing effect in wild type C. elegans. These peptides were then expressed in receptor mutants, and npr-7 mutants were found to block all effects while npr-22 mutants partially blocked the locomotion effect (FIG. 3, panes (B) & (C), and FIG. 14). Additionally, a mild effect on locomotion was observed in response to hs:NPFF; however, quantification revealed no significant difference between wild-type and hs:NPFF animals (FIG. 15).

TABLE 4
Comparison of the amino acid identity of C. elegans GPCRs, NPR-7
and NPR-77 with Drosophila and human GPCRs (alignment
performed with UniProt (www.uniprot.org))

| | NPR-7 | NPR-22a | NPR-22b |
|---|---|---|---|
| | (Q20067) | (Q9N324) | (Q59E83) |
| Human FF1 | 23.10% | 23.59% | 24 8% |
| (Q9GZQ8) | | | |
| Human FF2 | 18.40% | 20% | 20.2% |
| Q9Y5X5 | | | |
| Zebrafish | 20.9% | 23.7% | 23.7% |
| npffr-1l2 | | | |
| (FIQP75) | | | |
| Zebrafish | 22.10% | 23.8% | 23.7% |
| npffr-2.1 | | | |
| (A2AV71) | | | |
| Zebrafish | 21.30% | 22.9% | 22.3% |
| npffr-2.2 | | | |
| (FIQ764) | | | |
| Human | 23% | 24.70% | 23.8% |
| NPYR2 | | | |
| (P49146) | | | |

### Example 8

### Expression of Neuropeptides in Zebrafish

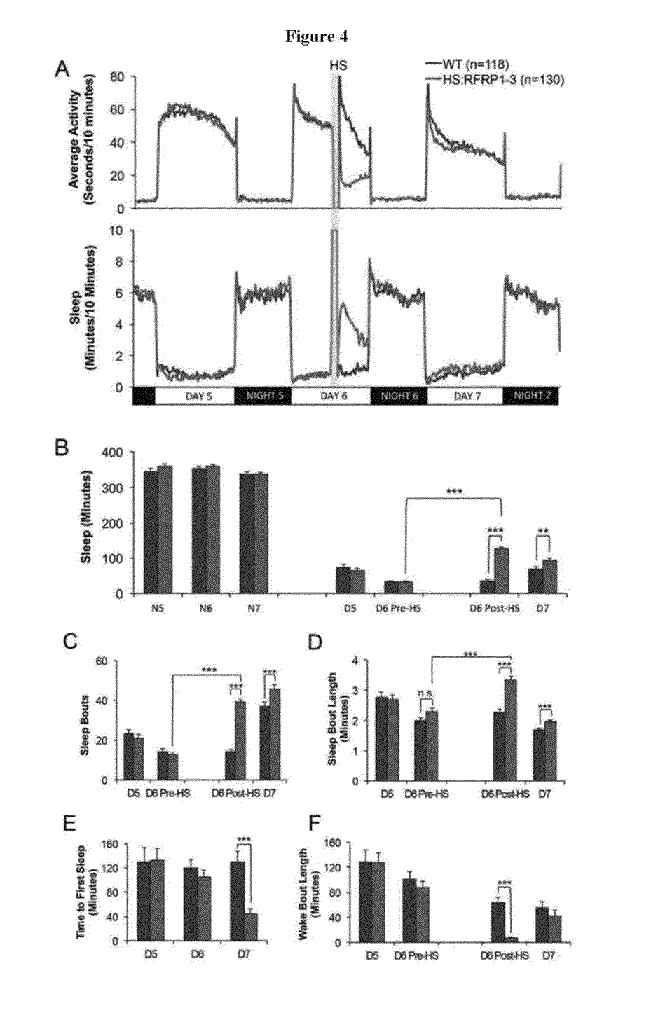

To determine whether RFRP neuropeptides regulate sleep/wake states in diurnal vertebrates, transgenic zebrafish were generated under the control of a heat-shock promoter that enabled the conditional overexpression of the RFRP precursor polypeptide (hs:RFRP), which becomes proteolytically processed into mature RFRP-1, RFRP-2, and RFRP-3 peptides (FIG. 16). The locomotor activity of the hs:RFRP and their wild type siblings was continuously monitored over several days using an automated video-tracking system (FIG. 4, panel (A) and FIG. 16). Prior to heatshock at 37° C., there was no statistically significant difference in locomotor activity or overall sleep quantity. Following heat-shock, however, a dramatic and significant reduction of locomotor activity was observed as well as a commensurate increase in overall sleep that was restricted to the day, but not night period (FIG. 4, panel (B)). This increased sleep was facilitated by changes within resting state (FIG. 4, panels (B), (C), & (D)), with an augmentation in the number of total sleep bouts and average sleep length observed in hs:RFRP versus wild type sibling controls. Furthermore, sleep latency, or time to first sleep, in the following day after heat-shock was significantly curtailed (FIG. 4, panel (E)).

These findings depict a previously unknown signaling pathway in vertebrate sleep regulation mediated by the FF1 receptor (e.g., NPFFR1) and its RFRP ligands. Further, zebrafish FF1 is likely the functional equivalent of nematode NPR-7, as ligands of both receptors induce sleep, and thus nematodes can be used for modeling NPFFR1 and similar GPCR-mediated sleep pathways.

### Example 9

### Statistics

Two-tail p-values were calculated using InStat software (GraphPad). Means were compared using an unpaired t-test with Welch's correction in the case of unequal variances.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method for regulating sleep in a subject, comprising administering to the subject a composition comprising an agent that modulates the activity of a G-protein coupled receptor (GCPR) selected from neuropeptide FF receptor 1 (NPFFR1), neuropeptide FF receptor 2 (NPFFR2), neuropeptide Y receptor type 1 (NPY1R), neuropeptide Y receptor type 2 (NPY2R), and tachykinin receptor (TACR).

2. The method of claim 1, wherein the GPCR is NPFFR1 or NPFFR2.

3. The method of claim 1, wherein the GPCR is NPFFR1.

4. The method of claim 1, wherein the agent is an agonist of the GPCR and the agent induces sleep in the subject.

5. The method of claim 4, wherein the subject has insomnia.

6. The method of claim 1, wherein the agent is an antagonist of the GPCR and the agent inhibits sleep in the subject.

7. The method of claim 6, wherein the subject has narcolepsy.

8. The method of claim 1, wherein the agent is a small molecule.

9. The method of claim 1, wherein administration comprises oral administration.

10. The method of claim 1, wherein the subject is a vertebrate.

11. The method of claim 10, wherein the subject is a human.

12. The method of claim 1, wherein regulating sleep in the subject comprises sedating the subject.

13. A method for identifying whether a compound induces sleep in a subject, comprising:
administering the compound to a test subject and monitoring the activity of the test subject, wherein the test subject comprises a genetic modification that reduces the expression of neuropeptide FF receptor 1 (NPFFR1), neuropeptide FF receptor 2 (NPFFR2), neuropeptide Y receptor type 1 (NPY1R), neuropeptide Y receptor type 2 (NPY2R), and/or tachykinin receptor (TACR), or an ortholog of any one of NPFFR1, NPFFR2, NPY1R, NPY2R and/or TACR;
administering the compound to a control subject and monitoring the activity of the control subject, wherein the control subject does not comprise the genetic modification; and
comparing the activity of the control subject and the test subject; and
identifying whether the compound induces sleep in the subject.

14. The method of claim 13, wherein the subject is a vertebrate.

15. The method of claim 14, wherein the subject is a zebrafish or mouse.

16. The method of claim 13, wherein the genetic modification is a knockdown or knockout mutation of a gene encoding NPFFR1, NPFFR2, NPY1R, NPY2R, or TACR, or an ortholog of any one of NPFFR1, NPFFR2, NPY1R, NPY2R and/or TACR.

17. The method of claim 13, wherein the activity is duration of sleep.

18. A method for inducing sleep in a subject, comprising administering to the subject a composition comprising a neuropeptide FF receptor 1 (NPFFR1) agonist.

19. The method of claim 18, wherein the subject is a human.

20. The method of claim 18, wherein the NPFFR1 agonist is a small molecule.
